(19) United States
(12) Patent Application Publication
(10) Pub. No.: US 2024/0141071 A1
(43) Pub. Date: May 2, 2024

(54) ANTIBODIES THAT BIND CD123 AND GAMMA-DELTA T CELL RECEPTORS

(71) Applicant: LAVA THERAPEUTICS N.V., Utrecht (NL)

(72) Inventors: Robertus Cornelis ROOVERS, Utrecht (NL); Johannes Jelle VAN DER VLIET, Utrecht (NL); David LUTJE HULSIK, Utrecht (NL); Paul Willem Henri Ida PARREN, Utrecht (NL); Jurjen Matthijs RUBEN, Utrecht (NL); Charlotte Merette MOUSSET, Utrecht (NL)

(21) Appl. No.: 18/278,956

(22) PCT Filed: Feb. 28, 2022
(86) PCT No.: PCT/EP2022/054993

§ 371 Date: Aug. 25, 2023

Related U.S. Application Data
(60) Provisional application No. 63/274,709, filed on Nov. 2, 2021.
(60) Provisional application No. 63/166,339, filed on Mar. 26, 2021.

(30) Foreign Application Priority Data
Feb. 26, 2021 (EP) 21159698.6
Nov. 29, 2021 (EP) 21211114.0

(51) Int. Cl. C07K 16/46; A61K 39/00; A61P 35/00

(52) U.S. Cl. CPC C07K 16/468; A61P 35/00; A61K 2039/505; C07K 2317/31; C07K 2317/565; C07K 2317/569

(57) ABSTRACT

The present invention relates to antibodies capable of binding human CD123 and capable of binding the Vδ2 chain of a human Vγ9Vδ2 T cell receptor. The invention further relates to pharmaceutical compositions comprising the antibodies of the invention and to uses of the antibodies of the invention for medical treatment.

### FIELD OF THE INVENTION

The present invention relates to novel multispecific antibodies capable of binding human CD123 and capable of binding the Vδ2 chain of a human Vγ9Vδ2 T cell receptor. The invention further relates to pharmaceutical compositions comprising the antibodies of the invention and to uses of the antibodies of the invention for medical treatment.

### BACKGROUND OF THE INVENTION

CD123, or the interleukin-3 (IL3) receptor alpha chain, is a membrane protein that transmits signaling by IL3, a cytokine involved in blood cell production. CD123 forms a heterodimer with the common beta chain CD131. CD123 is normally expressed on some types of blood cells, such as plasmacytoid dendritic cells or monocytes and by a subset of normal myeloid progenitors. However, CD123 is strongly overexpressed on leukemic stem cells of patients with acute myeloid leukemia. CD123 is therefore a potential therapeutic target in several hematologic malignancies, including acute myeloid leukemia.

Several bispecific CD123-CD3 T cell engaging antibodies have been described (Kuo et al. (2012) Protein Eng Des Set 10:561; Al-Hussaini et al. (2016) Blood 127:122). Bispecific T-cell engaging antibodies have a tumor target binding specificity and a T-cell binding specificity and thus boost efficacy by re-directing T-cell cytotoxicity to malignant cells, see e.g. Huehls et al. (2015) Immunol Cell Biol 93:290; Ellerman (2019) Methods, 154:102; de Bruin et al. (2017) Oncoimmunology 7(1):e1375641 and WO2015156673. However, results vary significantly. For example, in one study in which a CD3 binding moiety was combined with binding moieties against 8 different B-cell targets (CD20, CD22, CD24, CD37, CD70, CD79b, CD138 and HLA-DR), it was found that the bispecific antibodies targeting the different tumor targets showed strong variation in their capacity to induce target cell cytotoxicity and that cytotoxicity did not correlate with antigen expression levels. For example, CD3-based bispecific antibodies targeting HLA-DR or CD138 were not able to induce cytotoxicity, in spite of intermediate to high HLA-DR and CD138 expression levels (Engelberts et al. (2020) Ebiomedicine 52:102625). Few T-cell redirecting therapies have reached late-stage clinical development, possibly due to significant toxicity, manufacturing problems, immunogenicity, narrow therapeutic windows and low response. In particular, toxicity may occur when the T-cell engager includes a CD3 binding arm and result in uncontrolled, exaggerated, immune activation and cytokine release.

Thus, while significant progress has been made, there is still a need for novel CD123 targeting antibodies that are therapeutically effective yet have acceptable toxicity, as well as stability and manufacturability.

### SUMMARY OF THE INVENTION

The present invention provides novel antibodies for CD123-based therapy. Bispecific antibodies were constructed in which single-domain CD123-binding regions were combined with binding regions capable of binding the Vδ2 chain of a human Vγ9Vδ2 T cell receptor and thus engaging γδ T cells. Surprisingly, the bispecific antibodies were exceptionally potent in mediating activation of Vγ9Vδ2 T cells and inducing killing of CD123-expressing cell lines as well as patient-derived tumor cells in the presence of Vγ9Vδ2 T cells.

Accordingly, in a first aspect, the invention provides a multispecific antibody comprising a first antigen-binding region capable of binding human CD123 and a second antigen-binding region capable of binding the Vδ2 chain of a human Vγ9Vδ2 T cell receptor.

In a further main aspect, the invention provides an antibody comprising a first antigen-binding region capable of binding human CD123, wherein the first antigen-binding region is a single-domain antibody comprising:
(i) VH CDR1 sequence set forth in SEQ ID NO:2, the VH CDR2 sequence set forth in SEQ ID NO:3 and the VH CDR3 sequence set forth in SEQ ID NO:4, wherein preferably the first antigen-binding region comprises or consists of: a sequence selected from the group of sequences set forth in SEQ ID NO:1, 25 to 34, or a sequence having at least 90%, such as least 92%, e.g. at least 94%, such as at least 96%, e.g. at least 98% sequence identity to a sequence selected from the group of sequences set forth in SEQ ID NO:1, 25 to 34, or (ii) the VH CDR1 sequence set forth in SEQ ID NO:10, the VH CDR2 sequence set forth in SEQ ID NO:11 and the VH CDR3 sequence set forth in SEQ ID NO:12, wherein preferably the first antigen-binding region comprises or consists of: the sequence set forth in SEQ ID NO:9, or a sequence having at least 90%, such as least 92%, e.g. at least 94%, such as at least 96%, e.g. at least 98% sequence identity to the sequence set forth in SEQ ID NO:9.
   Further aspects and embodiments of the invention are described below.

### BRIEF DESCRIPTION OF THE DRAWINGS

FIG. 1: ELISA showing binding of all different bispecific VHHs to CD123, the Vγ9Vδ2 (GDT) TCR and BSA (as negative control). OD values are depicted; values are the mean of duplicate measurements; error bars indicate standard errors of the mean. A mono-valent anti-Vδ2 VHH was used as control for the TCR staining (“TCR control”) and a commercially available anti-CD123 antibody (“anti-CD123”) was used as control for the CD123 antigen coatings. “No AB” indicates a negative control without primary antibody.

FIG. 2: Specificity of binding of bispecific VHH using flow cytometry. The geometric mean of the fluorescence signal is plotted as a function of the antibody- and cell type used. A VHH recognizing EGFR that is endogenously expressed by 293F cells was included as positive control. A monovalent anti-Vδ2 VHH was used as negative control VHH.

FIG. 3: Determination of the apparent affinity of 1D2-5C8var1 for binding to CD123 using flow cytometry. A dilution series of purified 1D2-5C8var1 was tested for binding to 293F cells transiently expressing either CD123, or CD131, or both CD123 and -131. The geometric mean of the fluorescence intensity is plotted as a function of the antibody concentration used. The EC50 value was determined by curve fitting.

FIG. 4: Representative BLI analysis of 1D2-5C8var1 binding to CD123. The shift in light reflection (measured in nm) that is representative of the protein mass bound is plotted as a function of time. 0-300 seconds: association phase; 300-900 seconds: dissociation phase.

FIG. 5: C1R-neo target cell-dependent, 1D2-5C8var1-mediated Vγ9Vδ2 T cell activation. The percentage of CD3+-Vγ9+ T cells showing CD107A expression (degranulation) is plotted as a function of the concentration of antibody used. Experiments using two different donors are depicted.

FIG. 6: 1D2-5C8var1-induced, Vγ9Vδ2 T cell mediated C1R-neo target cell cytotoxicity. The percentage of living C1R-neo target cells is plotted as a function of the concentration of bispecific VHH used. Data obtained with two different donors of Vγ9Vδ2 T cells are depicted.

FIG. 7: Bispecific VHH-induced, Vγ9Vδ2 T cell mediated THP-1 target cell cytotoxicity. The percentage of living THP-1 target cells is plotted as a function of the concentration bispecific VHH used.

FIG. 8: Bispecific VHH-mediated Vγ9Vδ2 T cell activation and bispecific VHH mediated T cell induced lysis of a patient-derived primary AML sample. Upper panel: T cell activation as measured by CD107A expression. Lower panel: lysis of AML blasts by T cells in conjunction with bispecific VHH.

FIG. 9: HP-SEC profile of purified anti-CD123xVγ9Vδ2 TCR bispecific antibody 1D2-5C8var1(Y105F)-Fc.

FIG. 10: 1D2-5C8var1(Y105F)-Fc induces Vγ9Vδ2 T cell activation. A typical experiment is shown. The percentage of CD107a- (lysosomal-associated protein-1, or LAMP-1) positive Vγ9Vδ2 cells is depicted as a function of the concentration of compound used. EC50 values (in pM, determined by curve-fitting) are depicted below the graph. Data points are means of triplicate measurements; error bars represent standard deviations.

FIG. 11: 1D2-5C8var1(Y105F)-Fc induced T cell-mediated target cell lysis. A typical experiment is shown. The graph shows the percentage of target cells killed after 24 hours of co-culture as a function of the concentration of compound used. EC50 values (in pM, determined by curve-fitting) are depicted below the graph. Data points are means of triplicate measurements; error bars represent standard deviations.

FIG. 12: CD123 expression levels on plasmacytoid dendritic cells (upper panel) and on the THP-1 cell line (lower panel). A typical staining is shown. Histograms depicting non-stained cells, an isotype control staining (left overlapping histograms) and staining for CD123 (right peaks) are depicted. The number of events (Y-axis) is shown as function of the fluorescence intensity (X-axis).

FIG. 13: 1D2-5C8var1(Y105F)-Fc induces preferential killing of THP-1 cells compared to pDCs. A representative result is shown. The percentage of target cells killed is depicted as a function of the concentration of compound used per target cell population (i.e. THP-1 or pDC). EC50 values (in pM, determined by curve-fitting) are depicted below the graph. Data points are means of triplicate measurements; error bars represent standard deviations.

FIG. 14: Primary amino acid sequence of full length human CD123 (GenBank accession number NM_002183.4) (SEQ ID NO:23). The residues that were found cross-linked to the 1D2 antibody are bold and underlined. The residues in italics (flanked by found reactive residues) may also be part of the recognized epitope.

FIG. 15: C-alpha trace model of CD123 (the IL-3 receptor alpha chain: Broughton et al., 2018 2018 Nat Commun. 9: 386); the residues that were found cross-linked to the antibody are indicated. The membrane-spanning helix would be located to the left of the figure.

FIG. 16: Stress-induced changes as determined by measuring aggregates and fragments by (A) size exclusion chromatography detected by ultraviolet absorption (SEC-UV) and (B) capillary gel electrophoresis under denaturing (SDS) conditions (CE-SDS) and after reduction.

FIG. 17: (A) Degranulation analyzed after 4 hours by measuring the percentage CD107a (lysosomal-associated protein-1, or LAMP-1) positive cells via flow cytometry. (B) T cell activation analyzed by measuring the percentage CD25 positive cells. (C) cytotoxicity analyzed by determining the percentage live target cells after 24 hours via flow cytometry.

### DETAILED DESCRIPTION OF THE INVENTION

### Definitions

The term “human CD123”, when used herein, refers to the human CD123 protein, also termed interleukin-3 receptor alpha chain (GenBank accession number NM_002183.4, NCBI Reference Sequence: NP_002174.1). The sequence of human CD123 is set forth in SEQ ID NO:23. The IL3 receptor is a heterodimer of CD123 with CD131, a common beta chain (NCBI Reference Sequence: NP_000386.1). CD131 is set forth in SEQ ID NO:24.

The term “human Vδ2”, when used herein, refers to the rearranged δ2 chain of the Vγ9Vδ2-T cell receptor (TCR) (SEQ ID NO:48). UniProtKB-A0JD36 (A0JD36_HUMAN) gives an example of a variable TRDV2 sequence.

The term “human Vγ9”, when used herein, refers to the refers to the rearranged y9 chain of the Vγ9Vδ2-T cell receptor (TCR). UniProtKB-Q99603_HUMAN gives an example of a variable TRGV9 sequence.

The term “antibody” is intended to refer to an immunoglobulin molecule, a fragment of an immunoglobulin molecule, or a derivative of either thereof, which has the ability to specifically bind to an antigen under typical physiological conditions with a half-life of significant periods of time, such as at least about 30 minutes, at least about one hour, at least about two hours, at least about 8 hours, at least about 12 hours, about 24 hours or more, about 48 hours or more, about 3, 4, 5, 6, 7 or more days, etc., or any other relevant functionally-defined period (such as a time sufficient to induce, promote, enhance, and/or modulate a physiological response associated with antibody binding to the antigen and/or time sufficient for the antibody to recruit an effector activity). Antigen-binding regions which interact with an antigen may comprise variable regions of both the heavy and light chains of an immunoglobulin molecule or may comprise or consist of single-domain antigen-binding regions, for example a heavy chain variable region only. The constant regions of an antibody, if present, may mediate the binding of the immunoglobulin to host tissues or factors, including various cells of the immune system (such as effector cells and T cells) and components of the complement system such as C1q, the first component in the classical pathway of complement activation. In some embodiments, however, the Fc region of the antibody has been modified to become inert, “inert” means an Fc region which is at least not able to bind any Fey Receptors, induce Fc-mediated cross-linking of FcRs, or induce FcR-mediated cross-linking of target antigens via two Fc regions of individual antibodies. In a further embodiment, the inert Fc region is in addition not able to bind C1q. In one embodiment, the antibody contains mutations at positions 234 and 235 (Canfield and Morrison (1991) J Exp Med 173:1483), e.g. a Leu to Phe mutation at position 234 and a Leu to Glu mutation at position 235 (according to the EU-numbering, see below). In another embodiment, the antibody contains a Leu to Ala mutation at position 234, a Leu to Ala mutation at position 235 and a Pro to Gly mutation at position 329. In another embodiment, the antibody contains a Leu to Phe mutation at position 234, a Leu to Glu mutation at position 235 and an Asp to Ala at position 265.

The Fc region of an immunoglobulin is defined as the fragment of an antibody which would be typically generated after digestion of an antibody with papain which includes the two CH2-CH3 regions of an immunoglobulin and a connecting region, e.g. a hinge region. The constant domain of an antibody heavy chain defines the antibody isotype, e.g. IgG1, IgG2, IgG3, IgG4, IgA1, IgA2, IgM, IgD, or IgE. The Fc-region mediates the effector functions of antibodies with cell surface receptors called Fc receptors and proteins of the complement system.

The term “hinge region” as used herein is intended to refer to the hinge region of an immunoglobulin heavy chain. Thus, for example the hinge region of a human IgG1 antibody corresponds to amino acids 216-230 according to the EU numbering.

The term “CH2 region” or “CH2 domain” as used herein is intended to refer to the CH2 region of an immunoglobulin heavy chain. Thus, for example the CH2 region of a human IgG1 antibody corresponds to amino acids 231-340 according to the EU numbering. However, the CH2 region may also be any of the other subtypes as described herein.

The term “CH3 region” or “CH3 domain” as used herein is intended to refer to the CH3 region of an immunoglobulin heavy chain. Thus, for example the CH3 region of a human IgG1 antibody corresponds to amino acids 341-447 according to the EU numbering. However, the CH3 region may also be any of the other subtypes as described herein.

Reference to amino acid positions in the Fc region/Fc domain in the present invention is according to the EU-numbering (Edelman et al., Proc Natl Acad Sci USA. 1969 May;63(1):78-85; Kabat et al., Sequences of proteins of immunological interest. 5th Edition—1991 NIH Publication No. 91-3242).

As indicated above, the term antibody as used herein, unless otherwise stated or clearly contradicted by context, includes fragments of an antibody that retain the ability to specifically bind to the antigen. It has been shown that the antigen-binding function of an antibody may be performed by fragments of a full-length antibody. Examples of binding fragments encompassed within the term “antibody” include (i) a Fab′ or Fab fragment, i.e. a monovalent fragment consisting of the VL, VH, CL and CH1 domains, or a monovalent antibody as described in WO2007059782; (ii) F(ab′)2 fragments, i.e. bivalent fragments comprising two Fab fragments linked by a disulfide bridge at the hinge region; (iii) a Fd fragment consisting essentially of the VH and CH1 domains; and (iv) a Fv fragment consisting essentially of the VL and VH domains of a single arm of an antibody. Furthermore, although the two domains of the Fv fragment, VL and VH, are coded for by separate genes, they may be joined, using recombinant methods, by a synthetic linker that enables them to be made as a single protein chain in which the VL and VH regions pair to form monovalent molecules (known as single chain antibodies or single chain Fv (scFv), see for instance Bird et al., Science 242, 423-426 (1988) and Huston et al., PNAS USA 85, 5879-5883 (1988)). Such single-chain antibodies are encompassed within the term antibody unless otherwise indicated by context. The term antibody, unless specified otherwise, also includes polyclonal antibodies, monoclonal antibodies (mAbs), chimeric antibodies and humanized antibodies, and antibody fragments provided by any known technique, such as enzymatic cleavage, peptide synthesis, and recombinant techniques.

In some embodiments of the antibodies of the invention, the first antigen-binding region or the second antigen-binding region, or both, is a single-domain antibody. Single-domain antibodies (sdAb, also called Nanobody®, or VHH) are well known to the skilled person, see e.g. Hamers-Casterman et al. (1993) Nature 363:446, Roovers et al. (2007) Curr Opin Mol Ther 9:327 and Krah et al. (2016) Immunopharmacol Immunotoxicol 38:21. Single-domain antibodies comprise a single CDR1, a single CDR2 and a single CDR3. Examples of single-domain antibodies are variable fragments of heavy-chain-only antibodies, antibodies that naturally do not comprise light chains, single-domain antibodies derived from conventional antibodies, and engineered antibodies. Single-domain antibodies may be derived from any species including mouse, human, camel, llama, shark, goat, rabbit, and cow. For example, naturally occurring VHH molecules can be derived from antibodies raised in Camelidae species, for example in camel, dromedary, llama, alpaca and guanaco. Like a whole antibody, a single-domain antibody is able to bind selectively to a specific antigen. Single-domain antibodies may contain only the variable domain of an immunoglobulin chain, i.e. CDR1, CDR2 and CDR3 and framework regions.

The term “immunoglobulin” as used herein is intended to refer to a class of structurally related glycoproteins typically consisting of two pairs of polypeptide chains, one pair of light (L) chains and one pair of heavy (H) chains, all four potentially inter-connected by disulfide bonds, although some mammalian species do not product light chain and only make heavy-chain antibodies. The term “immunoglobulin heavy chain”, “heavy chain of an immunoglobulin” or “heavy chain” as used herein is intended to refer to one of the chains of an immunoglobulin. A heavy chain is typically comprised of a heavy chain variable region (abbreviated herein as VH) and a heavy chain constant region (abbreviated herein as CH) which defines the isotype of the immunoglobulin. The heavy chain constant region typically is comprised of three domains, CH1, CH2, and CH3. The heavy chain constant region further comprises a hinge region. Within the structure of the immunoglobulin (e.g. IgG), the two heavy chains are inter-connected via disulfide bonds in the hinge region. Equally to the heavy chains, each light chain is typically comprised of several regions; a light chain variable region (VL) and a light chain constant region (CL). Furthermore, the VH and VL regions may be subdivided into regions of hypervariability (or hypervariable regions which may be hypervariable in sequence and/or form structurally defined loops), also termed complementarity determining regions (CDRs), interspersed with regions that are more conserved, termed framework regions (FRs). Each VH and VL is typically composed of three CDRs and four FRs, arranged from amino-terminus to carboxy-terminus in the following order: FR1, CDR1, FR2, CDR2, FR3, CDR3, FR4. CDR sequences may be determined by use of various methods, e.g. the methods provided by Choitia and Lesk (1987) J. Mol. Biol. 196:901 or Kabat et al. (1991) Sequence of protein of immunological interest, fifth edition. NIH publication. Various methods for CDR determination and amino acid numbering can be compared on www.abysis.org (UCL).

The term “isotype” as used herein, refers to the immunoglobulin (sub)class (for instance IgG1, IgG2, IgG3, IgG4, IgD, IgA, IgE, or IgM) or any allotype thereof, such as IgG1m(za) and IgG1m(f) that is encoded by heavy chain constant region genes. Each heavy chain isotype can be combined with either a kappa (κ) or lambda (λ) light chain. An antibody of the invention can possess any isotype.

The term “parent antibody”, is to be understood as an antibody which is identical to an antibody according to the invention, but wherein the parent antibody does not have one or more of the specified mutations. A “variant” or “antibody variant” or a “variant of a parent antibody” of the present invention is an antibody molecule which comprises one or more mutations as compared to a “parent antibody”. Amino acid substitutions may exchange a native amino acid for another naturally-occurring amino acid, or for a non-naturally-occurring amino acid derivative. The amino acid substitution may be conservative or non-conservative. In the context of the present invention, conservative substitutions may be defined by substitutions within the classes of amino acids reflected in one or more of the following three tables:

### Amino Acid Residue Classes for Conservative Substitutions

| | |
|---|---|
| Acidic Residues | Asp (D) and Glu (E) |
| Basic Residues | Lys (K), Arg (R), and His (H) |
| Hydrophilic Uncharged Residues | Ser (S), Thr (T), Asn (N), and |
| | Gln (Q) |
| Aliphatic Uncharged Residues | Gly (G), Ala (A), Val (V), Leu (L), and |
| | Ile (I) |
| Non-polar Uncharged Residues | Cys (C), Met (M), and Pro (P) |
| Aromatic Residues | Phe (F), Tyr (Y), and Trp (W) |

### Alternative Conservative Amino Acid Residue Substitution Classes

| | | | |
|---|---|---|---|
| 1 | A | S | T |
| 2 | D | E | |
| 3 | N | Q | |
| 4 | R | K | |
| 5 | I | L | M |
| 6 | F | Y | W |

### Alternative Physical and Functional Classifications of Amino Acid Residues

| | |
|---|---|
| Alcohol group-containing residues | S and T |
| Aliphatic residues | I, L, V, and M |
| Cycloalkenyl-associated residues | F, H, W, and Y |
| Hydrophobic residues | A, C, F, G, H, I, L, M, |
| | R, T, V, W, and Y |
| Negatively charged residues | D and E |
| Polar residues | C, D, E, H, K, N, Q, R, S, and T |
| Positively charged residues | H, K, and R |
| Small residues | A, C, D, G, N, P, S, T, and V |
| Very small residues | A, G, and S |
| Residues involved in turn formation | A, C, D, E, G, H, K, N, |
| | Q, R, S, P, and T |
| Flexible residues | Q, T, K, S, G, N, D, E, and R |

In the context of the present invention, a substitution in a variant is indicated as:
Original amino acid-position-substituted amino acid;

The three-letter code, or one letter code, are used, including the codes Xaa and X to indicate amino acid residue. Accordingly, the notation “T366W” means that the variant comprises a substitution of threonine with tryptophan in the variant amino acid position corresponding to the amino acid in position 366 in the parent antibody.

Furthermore, the term “a substitution” includes a substitution into any one of the other nineteen natural amino acids, or into other amino acids, such as non-natural amino acids. For example, a substitution of amino acid T in position 366 includes each of the following substitutions: 366A, 366C, 366D, 366G, 366H, 366F, 366I, 366K, 366L, 366M, 366N, 366P, 366Q, 366R, 366S, 366E, 366V, 366W, and 366Y.

The term “full-length antibody” when used herein, refers to an antibody which contains all heavy and light chain constant and variable domains corresponding to those that are normally found in a wild-type antibody of that isotype.

The term “chimeric antibody” refers to an antibody wherein the variable region is derived from a non-human species (e.g. derived from rodents) and the constant region is derived from a different species, such as human. Chimeric antibodies may be generated by genetic engineering. Chimeric monoclonal antibodies for therapeutic applications are developed to reduce antibody immunogenicity.

The term “humanized antibody” refers to a genetically engineered non-human antibody, which contains human antibody constant domains and non-human variable domains modified to contain a high level of sequence homology to human variable domains. This can be achieved by grafting of the six non-human antibody complementarity-determining regions (CDRs), which together form the antigen binding site, onto a homologous human acceptor framework region (FR). In order to fully reconstitute the binding affinity and specificity of the parental antibody, the substitution of framework residues from the parental antibody (i.e. the non-human antibody) into the human framework regions (back-mutations) may be required. Structural homology modeling may help to identify the amino acid residues in the framework regions that are important for the binding properties of the antibody. Thus, a humanized antibody may comprise non-human CDR sequences, primarily human framework regions optionally comprising one or more amino acid back-mutations to the non-human amino acid sequence, and, optionally, fully human constant regions. Optionally, additional amino acid modifications, which are not necessarily back-mutations, may be introduced to obtain a humanized antibody with preferred characteristics, such as affinity and biochemical properties. Humanization of non-human therapeutic antibodies is performed to minimize its immunogenicity in man while such humanized antibodies at the same time maintain the specificity and binding affinity of the antibody of non-human origin.

The term “multispecific antibody” refers to an antibody having specificities for at least two different, such as at least three, typically non-overlapping, epitopes, due to the presence of two or more antigen-binding regions. Such epitopes may be on the same or on different target antigens. If the epitopes are on different targets, such targets may be on the same cell or different cells or cell types.

The term “bispecific antibody” refers to an antibody having specificities for two different, typically non-overlapping, epitopes, due to the presence of two antigen-binding regions. Such epitopes may be on the same or different targets. If the epitopes are on different targets, such targets may be on the same cell or different cells or cell types.

Examples of different classes of bispecific antibodies include but are not limited to (i) IgG-like molecules with complementary CH3 domains to force heterodimerization; (ii) recombinant IgG-like dual targeting molecules, wherein the two sides of the molecule each contain the Fab fragment or part of the Fab fragment of at least two different antibodies; (iii) IgG fusion molecules, wherein full length IgG antibodies are fused to extra Fab fragment or parts of Fab fragment; (iv) Fc fusion molecules, wherein single chain Fv molecules or stabilized diabodies are fused to heavy-chain constant-domains, Fc-regions or parts thereof; (v) Fab fusion molecules, wherein different Fab-fragments are fused together, fused to heavy-chain constant-domains, Fc-regions or parts thereof; and (vi) scFv-and diabody-based and heavy chain antibodies (e.g., domain antibodies, Nanobodies®) wherein different single chain Fv molecules or different diabodies or different heavy-chain antibodies (e.g. domain antibodies, Nanobodies®) are fused to each other or to another protein or carrier molecule fused to heavy-chain constant-domains, Fc-regions or parts thereof.

Examples of IgG-like molecules with complementary CH3 domains molecules include but are not limited to the Triomab® (Trion Pharma/Fresenius Biotech), the Knobs-into-Holes (Genentech), CrossMAbs (Roche) and the electrostatically-matched (Amgen, Chugai, Oncomed), the LUZ-Y (Genentech, Wranik et al. J. Biol. Chem. 2012, 287(52): 43331-9, doi: 10.1074/jbc.M112.397869. Epub 2012 Nov 1), DIG-body and PIG-body (Pharmabcine, WO2010134666, WO2014081202), the Strand Exchange Engineered Domain body (SEEDbody)(EMD Serono), the Biclonics (Merus, WO2013157953), FcΔAdp (Regeneron), bispecific IgG1 and IgG2 (Pfizer/Rinat), Azymetric scaffold (Zymeworks/Merck,), mAb-Fv (Xencor), bivalent bispecific antibodies (Roche, WO2009080254) and DuoBody® molecules (Genmab).

Examples of recombinant IgG-like dual targeting molecules include, but are not limited, to Dual Targeting (DT)-Ig (GSK/Domantis, WO2009058383), Two-in-one Antibody (Genentech, Bostrom, et al 2009. Science 323, 1610-1614), Cross-linked Mabs (Karmanos Cancer Center), mAb2 (F-Star), Zybodies™ (Zyngenia, LaFleur et al. MAbs. 2013 Mar-Apr;5(2):208-18), approaches with common light chain, κλBodies (NovImmune, WO2012023053) and CovX-body® (CovX/Pfizer, Doppalapudi, V. R., et al 2007. Bioorg. Med. Chem. Lett. 17,501-506).

Examples of IgG fusion molecules include but are not limited to Dual Variable Domain (DVD)-Ig (Abbott), Dual domain double head antibodies (Unilever; Sanofi Aventis), IgG-like Bispecific (ImClone/Eli Lilly, Lewis et al. Nat Biotechnol. 2014 Feb;32(2):191-8), Ts2Ab (MedImmune/AZ, Dimasi et al. J Mol Biol. 2009 Oct 30;393(3):672-92) and BsAb (Zymogenetics, WO2010111625), HERCULES (Biogen Idec), scFv fusion (Novartis), scFv fusion (Changzhou Adam Biotech Inc) and TvAb (Roche).

Examples of Fc fusion molecules include but are not limited to scFv/Fc Fusions (Academic Institution, Pearce et al Biochem Mol Biol Int. 1997 Sep;42(6):1179), SCORPION (Emergent BioSolutions/Trubion, Blankenship J W, et al. AACR 100th Annual meeting 2009 (Abstract #5465); Zymogenetics/BMS, WO2010111625), Dual Affinity Retargeting Technology (Fc-DART™) (MacroGenics) and Dual(ScFv)2-Fab (National Research Center for Antibody Medicine-China).

Examples of Fab fusion bispecific antibodies include but are not limited to F(ab)′2 (Medarex/AMGEN), Dual-Action or Bis-Fab (Genentech), Dock-and-Lock® (DNL) (ImmunoMedics), Bivalent Bispecific (Biotecnol) and Fab-Fv (UCB-Celltech).

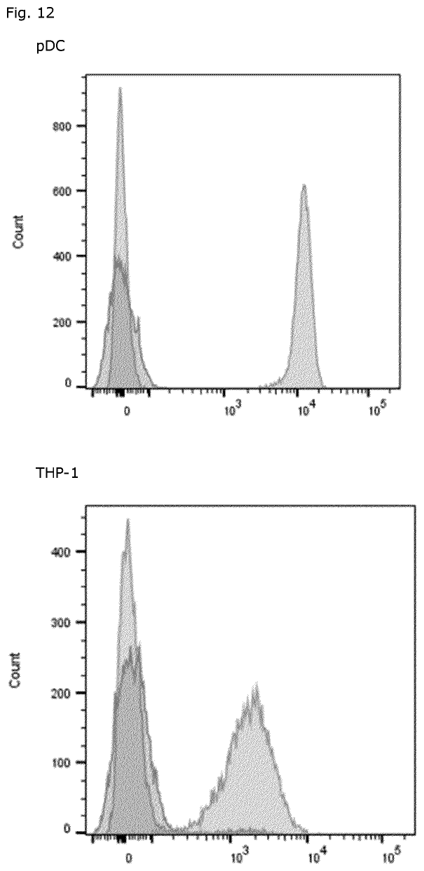

Examples of scFv-, diabody-based and domain antibodies include but are not limited to Bispecific T Cell Engager (BITE®) (Micromet, Tandem Diabody (Tandab) (Affimed), Dual Affinity Retargeting Technology (DART™) (MacroGenics), Single-chain Diabody (Academic, Lawrence FEBS Lett. 1998 Apr 3;425(3):479-84), TCR-like Antibodies (AIT, ReceptorLogics), Human Serum Albumin ScFv Fusion (Merrimack, WO2010059315) and COMBODY molecules (Epigen Biotech, Zhu et al. Immunol Cell Biol. 2010 Aug;88(6):667-75), dual targeting nanobodies® (Ablynx, Hmila et al., FASEB J. 2010), dual targeting heavy chain only domain antibodies. In some embodiments, the multispecific antibody of the invention is in a VHH-Fc format, i.e. the antibody comprises two or more single-domain antigen-binding regions that are linked to each other via a human Fc region dimer. In this format, each single-domain antigen-binding region is fused to an Fc region polypeptide and the two fusion polypeptides form a dimeric bispecific antibody via disulfide bridges in the hinge region. Such constructs typically do not contain full, or any, CH1 or light chain sequences. FIG. 12B of WO06064136 provides an illustration of an example of this embodiment.

In the context of antibody binding to an antigen, the terms “binds” or “specifically binds” refer to the binding of an antibody to a predetermined antigen or target (e.g. human CD123 or Vδ2) to which binding typically is with an affinity corresponding to a K_{D} of about 10⁻⁶ M or less, e.g. 10⁻⁷ M or less, such as about 10⁻⁸ M or less, such as about 10⁻⁹ or less, about 10⁻¹⁰ M or less, or about 10⁻¹¹ M or even less, e.g. when determined using flow cytometry as described in the Examples herein. Alternatively, K_{D} values can be determined using for instance surface plasmon resonance (SPR) technology in a BIAcore T200 or bio-layer interferometry (BLI) in an Octet RED96 instrument using the antigen as the ligand and the binding moiety or binding molecule as the analyte. Specific binding means that the antibody binds to the predetermined antigen with an affinity corresponding to a K_{D} that is at least ten-fold lower, such as at least 100-fold lower, for instance at least 1,000 fold lower, such as at least 10,000 fold lower, for instance at least 100,000 fold lower than its affinity for binding to a non-specific antigen (e.g., BSA, casein) other than the predetermined antigen or a closely-related antigen. The degree with which the affinity is lower is dependent on the K_{D} of the binding moiety or binding molecule, so that when the K_{D} of the binding moiety or binding molecule is very low (that is, the binding moiety or binding molecule is highly specific), then the degree with which the affinity for the antigen is lower than the affinity for a non-specific antigen may be at least 10,000-fold. The term “K_{D}” (M), as used herein, refers to the dissociation equilibrium constant of a particular interaction between the antigen and the binding moiety or binding molecule.

In the context of the present invention, “competition” or “able to compete” or “competes” refers to any detectably significant reduction in the propensity for a particular binding molecule (e.g. a CD123 antibody) to bind a particular binding partner (e.g. CD123) in the presence of another molecule (e.g. a different CD123 antibody) that binds the binding partner. Typically, competition means an at least about 25 percent reduction, such as an at least about 50 percent, e.g. an at least about 75 percent, such as an at least 90 percent reduction in binding, caused by the presence of another molecule, such as an antibody, as determined by, e.g., ELISA analysis or flow cytometry using sufficient amounts of the two or more competing molecules, e.g. antibodies. Additional methods for determining binding specificity by competitive inhibition may be found in for instance Harlow et al., Antibodies: A Laboratory Manual, Cold Spring Harbor Laboratory Press, Cold Spring Harbor, N.Y., 1988), Colligan et al., eds., Current Protocols in Immunology, Greene Publishing Assoc, and Wiley InterScience N. Y., (1992, 1993), and Muller, Meth. Enzymol. 92, 589-601 (1983)). In one embodiment, the antibody of the present invention binds to the same epitope on CD123 as antibody 1D2 or 1A3 and/or to the same epitope on Vδ2 as antibody 5C8, 6H4, 6C1, 5D3 (WO2015156673) or 5C8var1 (WO2020060405). The epitope of 5C8 has been determined to include residues S33, S43 and K45 (SEQ ID NO:48). The epitope of 6H4 has been determined to include residues R139, K152, 5189 and S191 (SEQ ID NO:48). There are several methods available for mapping antibody epitopes on target antigens known in the art, including but not limited to: crosslinking coupled mass spectrometry, allowing identification of peptides that are part of the epitope, and X-ray crystallography identifying individual residues on the antigen that form the epitope. Epitope residues can be determined as being all amino acid residues with at least one atom less than or equal to 5 Å from the antibody. 5 Å was chosen as the epitope cutoff distance to allow for atoms within a van der Waals radius plus a possible water-mediated hydrogen bond. Next, epitope residues can be determined as being all amino acid residues with at least one atom less than or equal to 8 Å. Less than or equal to 8 Å is chosen as the epitope cutoff distance to allow for the length of an extended arginine amino acid. Crosslinking coupled mass spectrometry begins by binding the antibody and the antigen with a mass labeled chemical crosslinker. Next the presence of the complex is confirmed using high mass MALDI detection. Because after crosslinking chemistry the Ab/Ag complex is extremely stable, many various enzymes and digestion conditions can be applied to the complex to provide many different overlapping peptides. Identification of these peptides is performed using high resolution mass spectrometry and MS/MS techniques. Identification of the crosslinked peptides is determined using mass tag linked to the cross-linking reagents. After MS/MS fragmentation and data analysis, peptides that are crosslinked and are derived from the antigen are part of the epitope, while peptides derived from the antibody are part of the paratope. All residues between the most N- and C-terminal crosslinked residue from the individual crosslinked peptides found are considered to be part of the epitope or paratope.

The terms “first” and “second” antigen-binding regions when used herein do not refer to their orientation/position in the antibody, i.e. they have no meaning with regard to the N- or C-terminus. The terms “first” and “second” only serve to correctly and consistently refer to the two different antigen-binding regions in the claims and the description.

“Capable of binding human CD123” means that the antibody can bind human CD123 as a separate molecule and/or as part of a CD123/CD131 complex. However, the antibody will not bind to CD131 as a separate molecule.

“Capable of binding the Vδ2 chain of a Vγ9Vδ2-TCR” means that the antibody can bind the Vδ2 chain as a separate molecule and/or as part of a Vγ9Vδ2-TCR. However, the antibody will not bind to the Vγ9 chain as a separate molecule.

“% sequence identity”, when used herein, refers to the number of identical nucleotide or amino acid positions shared by different sequences (i.e., % identity=# of identical positions/total # of positions x 100), taking into account the number of gaps, and the length of each gap, which need to be introduced for optimal alignment. The percent identity between two nucleotide or amino acid sequences may e.g. be determined using the algorithm of E. Meyers and W. Miller, Comput. Appl. Biosci 4, 11-17 (1988) which has been incorporated into the ALIGN program (version 2.0), using a PAM120 weight residue table, a gap length penalty of 12 and a gap penalty of 4.

### Further Aspects and Embodiments of the Invention

As described above, in a first main aspect, the invention relates to a multispecific antibody comprising a first antigen-binding region capable of binding human CD123 and a second antigen-binding region capable of binding the Vδ2 chain of a human Vγ9Vδ2 T cell receptor.

In one embodiment, the multispecific antibody is a bispecific antibody. In another embodiment, the multispecific antibody is a trispecific antibody. In another embodiment, the first antigen-binding region is a single-domain antibody, for example a single-domain antibody which consists of a heavy chain variable region. In another embodiment, the second antigen-binding region is a single-domain antibody, for example a single-domain antibody which consists of a heavy chain variable region. In a further embodiment, both the first antigen-antigen binding region and the second antigen-binding region are single-domain antibodies, for example single-domain antibodies which each consist of a heavy chain variable region.

In a further embodiment, the multispecific antibody is a bispecific antibody, wherein the first antigen-binding region is a single-domain antibody and the second antigen-binding region is a single-domain antibody. Said bispecific antibody may optionally comprise further sequences, such as a linker and/or an immunoglobulin Fc region.

In one embodiment, the multispecific antibody competes (i.e. is able to compete) for binding to human CD123 with an antibody having the sequence set forth in SEQ ID NO:1, preferably wherein the multispecific antibody binds the same epitope on human CD123 as an antibody having the sequence set forth in SEQ ID NO:1. In one embodiment, the multispecific antibody binds to an epitope comprising one or more residues in the region from S203 to R273, such as an epitope fully comprised within the region from S203 to R273, determined as described in Example 11 herein.

In another embodiment, the multispecific antibody binds to an epitope on human CD123 which comprises one or more residues in the region S203 to T214 and one or more residues in the region H221 to K227 and one or more residues in the region Y238 to K244 and one or more residues in the region Y268 to R273 (FIG. 14).

In another embodiment, the multispecific antibody binds to an epitope on human CD123 which comprises one, more of all of the residues S203, T209, T214, H221, H225, K227, Y238, K244, Y268, T269 and R273 (FIG. 14).

In one embodiment, the first antigen-binding region comprises the VH CDR1 sequence set forth in SEQ ID NO:2, the VH CDR2 sequence set forth in SEQ ID NO:3 and the VH CDR3 sequence set forth in SEQ ID NO:4.

In one embodiment, in SEQ ID NO:2, X₁ is G. In another embodiment, X₁ is S.

In one embodiment, in SEQ ID NO:3, X₂ is A. In another embodiment, X₂ is T.

In one embodiment, in SEQ ID NO:4, X₃ is Y. In another embodiment, X₃ is F.

In one embodiment, X₁ is G, X₂ is A and X₃ is Y.

In another embodiment, X₁ is G, X₂ is A and X₃ is F.

In another embodiment, X₁ is G, X₂ is T and X₃ is Y.

In another embodiment, X₁ is G, X₂ is T and X₃ is F.

In one embodiment, X₁ is S, X₂ is A and X₃ is Y.

In another embodiment, X₁ is S, X₂ is A and X₃ is F.

In another embodiment, X₁ is S, X₂ is T and X₃ is Y.

In another embodiment, X₁ is S, X₂ is T and X₃ is F.

In one embodiment, the first antigen-binding region comprises or consists of: the sequence set forth in SEQ ID NO:1, or a sequence having at least 90%, such as least 92%, e.g. at least 94%, such as at least 96%, e.g. at least 98% sequence identity to the sequence set forth in SEQ ID NO:1.

In another embodiment, the first antigen-binding region comprises or consists of a sequence selected from the group of sequences set forth in SEQ ID NO:25 to 34, or a sequence having at least 90%, such as least 92%, e.g. at least 94%, such as at least 96%, e.g. at least 98% sequence identity to a sequence selected from the group of sequences set forth in SEQ ID NO:25 to 34.

In another embodiment, the multispecific antibody competes for binding to human CD123 with an antibody with an antibody having the sequence set forth in SEQ ID NO:9, preferably wherein the multispecific antibody binds the same epitope on human CD123 as an antibody having the sequence set forth in SEQ ID NO:9.

In one embodiment, the multispecific antibody binds to an epitope comprising one or more residues in the region from H225 to T267, such as an epitope fully comprised within the region from H225 to T267, determined as described in Example 11 herein.

In another embodiment, the multispecific antibody binds to an epitope on human CD123 which comprises one or more residues in the region H225 and R234 and one or more residues in the region T251 to T267.

In another embodiment, the multispecific antibody binds to an epitope on human CD123 which comprises one, more of all of the residues H225, H231, R234, T251, R255 and T267.

In one embodiment, the first antigen-binding region comprises the VH CDR1 sequence set forth in SEQ ID NO:10, the VH CDR2 sequence set forth in SEQ ID NO:11 and the VH CDR3 sequence set forth in SEQ ID NO:12.

In one embodiment, the first antigen-binding region comprises or consists of: the sequence set forth in SEQ ID NO:9, or a sequence having at least 90%, such as least 92%, e.g. at least 94%, such as at least 96%, e.g. at least 98% sequence identity to the sequence set forth in SEQ ID NO:9.

As described above, the multispecific antibody of the invention comprises a second antigen-binding region capable of binding the Vδ2 chain of a human Vγ9Vδ2-T cell receptor. Vδ2 is part of the delta chain of the Vγ9Vδ2-TCR. An antibody capable of binding to human Vδ2 may bind an epitope that is entirely located within the Vδ2 region or bind an epitope that is a combination of residues in Vδ2 region and the constant region of the delta chain. In one embodiment, the multispecific antibody is able to activate human Vγ9Vδ2 T cells. The activation of the Vγ9Vδ2 T cells may be measured through gene-expression and/or (surface) marker expression (e.g., activation markers, such as CD25, CD69, or CD107a) and/or secretory protein (e.g., cytokines or chemokines) profiles. In a preferred embodiment, the multispecific antibody is able to induce activation (e.g. upregulation of CD69 and/or CD25 expression) resulting in degranulation marked by an increase in CD107a expression (see the Examples herein) and/or cytokine production (e.g. TNFα, IFNγ) by Vγ9Vδ2 T cells. Preferably, a multispecific antibody of the present invention is able to increase the number of cells positive for CD107a at least 2-fold, such as at least 5-fold, when tested as described in the Examples herein. In another preferred embodiment, the multispecific antibody of the invention has an EC50 value for increasing the percentage of CD107a positive cells of 50 pM or less, such as 25 pM or less, e.g. 20 pM or less, such as 15 pM or less, e.g. 10 pM or less, when tested using Vγ9Vδ2 T cells and C1r-neo target cells as described herein in the Examples.

Several antibodies which bind to Vδ2 have been described in WO2015156673 and their antigen-binding regions or at least the CDR sequences thereof can be incorporated in the multispecific antibody of the invention.

In one embodiment, the multispecific antibody competes for binding to human Vδ2 with an antibody having the sequence set forth in SEQ ID NO:17 wherein X₄ is Y.

In a further embodiment, the multispecific antibody binds the same epitope on human Vδ2 as an antibody having the sequence set forth in SEQ ID NO:17.

In one embodiment, the multispecific antibody competes for binding to human Vδ2 with an antibody having the sequence set forth in SEQ ID NO:36, preferably the multispecific antibody binds the same epitope on human Vδ2 as an antibody having the sequence set forth in SEQ ID NO:36.

In one embodiment, the multispecific antibody competes for binding to human Vδ2 with an antibody having the sequence set forth in SEQ ID NO:37, preferably the multispecific antibody binds the same epitope on human Vδ2 as an antibody having the sequence set forth in SEQ ID NO:37.

In one embodiment, the multispecific antibody competes for binding to human Vδ2 with an antibody having the sequence set forth in SEQ ID NO:38, preferably the multispecific antibody binds the same epitope on human Vδ2 as an antibody having the sequence set forth in SEQ ID NO:38.

In one embodiment of the multispecific antibody of the invention, the second antigen-binding region comprises the VH CDR1 sequence set forth in SEQ ID NO:18, the VH CDR2 sequence set forth in SEQ ID NO:19 and the VH CDR3 sequence set forth in SEQ ID NO:20. In one embodiment, X₄ in SEQ ID NO:20 is Y. In another embodiment, X₄ in SEQ ID NO:20 is F. In another embodiment, X₄ in SEQ ID NO:20 is S.

In one embodiment of the multispecific antibody of the invention, the second antigen-binding region comprises the VH CDR1 sequence set forth in SEQ ID NO:40 and the VH CDR3 sequence set forth in SEQ ID NO:41, preferably the second antigen-binding region comprises or consists of the sequence set forth in SEQ ID NO:36, or a sequence having at least 90%, such as least 92%, e.g. at least 94%, such as at least 96%, e.g. at least 98% sequence identity to the sequence set forth in SEQ ID NO:36,

In one embodiment of the multispecific antibody of the invention, the second antigen-binding region comprises the VH CDR1 sequence set forth in SEQ ID NO:42, the VH CDR2 sequence set forth in SEQ ID NO:43 and the VH CDR3 sequence set forth in SEQ ID NO:44, preferably the second antigen-binding region comprises or consists of the sequence set forth in SEQ ID NO:37, or a sequence having at least 90%, such as least 92%, e.g. at least 94%, such as at least 96%, e.g. at least 98% sequence identity to the sequence set forth in SEQ ID NO:37,

In one embodiment of the multispecific antibody of the invention, the second antigen-binding region comprises the VH CDR1 sequence set forth in SEQ ID NO:45, the VH CDR2 sequence set forth in SEQ ID NO:46 and the VH CDR3 sequence set forth in SEQ ID NO:47, preferably the second antigen-binding region comprises or consists of the sequence set forth in SEQ ID NO:38, or a sequence having at least 90%, such as least 92%, e.g. at least 94%, such as at least 96%, e.g. at least 98% sequence identity to the sequence set forth in SEQ ID NO:38.

In one embodiment of the multispecific antibody of the invention, the second antigen-binding region is humanized.

In a further embodiment, the second antigen-binding region comprises or consists of the sequence set forth in SEQ ID NO:17, or a sequence having at least 90%, such as least 92%, e.g. at least 94%, such as at least 96%, e.g. at least 98% sequence identity to the sequence set forth in SEQ ID NO:17. In one embodiment, X₄ in SEQ ID NO: 17 is Y. In another embodiment, X₄ in SEQ ID NO:17 is F. In another embodiment, X₄ in SEQ ID NO:17 is S.

In a preferred embodiment of the multispecific antibody of the invention,
(i) the first antigen-binding region comprises the VH CDR1 sequence set forth in SEQ ID NO:2, the VH CDR2 sequence set forth in SEQ ID NO:3 and the VH CDR3 sequence set forth in SEQ ID NO:4 and the second antigen-binding region comprises the VH CDR1 sequence set forth in SEQ ID NO:18, the VH CDR2 sequence set forth in SEQ ID NO:19 and the VH CDR3 sequence set forth in SEQ ID NO:20, or
(ii) the first antigen-binding region comprises the VH CDR1 sequence set forth in SEQ ID NO:10, the VH CDR2 sequence set forth in SEQ ID NO:11 and the VH CDR3 sequence set forth in SEQ ID NO:12 and the second antigen-binding region comprises the VH CDR1 sequence set forth in SEQ ID NO:18, the VH CDR2 sequence set forth in SEQ ID NO:19 and the VH CDR3 sequence set forth in SEQ ID NO:20.
   In further preferred embodiments of the multispecific antibody of the invention,
(i) the first antigen-binding region comprises or consists of the sequence set forth in SEQ ID NO:1 and the second antigen-binding region comprises or consists of the sequence set forth in SEQ ID NO:17, wherein optionally X₄ is Y,
or
(ii) the first antigen-binding region comprises or consists of the sequence set forth in SEQ ID NO:9 and the second antigen-binding region comprises or consists of the sequence set forth in SEQ ID NO:17, wherein optionally X₄ is Y,
or
(iii) the first antigen-binding region comprises or consists of the sequence set forth in SEQ ID NO:25 and the second antigen-binding region comprises or consists of the sequence set forth in SEQ ID NO:17, wherein optionally X₄ is Y,
or
(iv) the first antigen-binding region comprises or consists of the sequence set forth in SEQ ID NO:26 and the second antigen-binding region comprises or consists of the sequence set forth in SEQ ID NO:17, wherein optionally X₄ is Y,
or
(v) the first antigen-binding region comprises or consists of the sequence set forth in SEQ ID NO:27 and the second antigen-binding region comprises or consists of the sequence set forth in SEQ ID NO:17, wherein optionally X₄ is Y,
or
(vi) the first antigen-binding region comprises or consists of the sequence set forth in SEQ ID NO:28 and the second antigen-binding region comprises or consists of the sequence set forth in SEQ ID NO:17, wherein optionally X₄ is Y,
or
(vii) the first antigen-binding region comprises or consists of the sequence set forth in SEQ ID NO:29 and the second antigen-binding region comprises or consists of the sequence set forth in SEQ ID NO:17, wherein optionally X₄ is Y,
or
(viii) the first antigen-binding region comprises or consists of the sequence set forth in SEQ ID NO:30 and the second antigen-binding region comprises or consists of the sequence set forth in SEQ ID NO:17, wherein optionally X₄ is Y,
or
(ix) the first antigen-binding region comprises or consists of the sequence set forth in SEQ ID NO:31 and the second antigen-binding region comprises or consists of the sequence set forth in SEQ ID NO:17, wherein optionally X₄ is Y,
or
(x) the first antigen-binding region comprises or consists of the sequence set forth in SEQ ID NO:32 and the second antigen-binding region comprises or consists of the sequence set forth in SEQ ID NO:17, wherein optionally X₄ is Y,
or
(xi) the first antigen-binding region comprises or consists of the sequence set forth in SEQ ID NO:33 and the second antigen-binding region comprises or consists of the sequence set forth in SEQ ID NO:17, wherein optionally X₄ is Y,
or
(xii) the first antigen-binding region comprises or consists of the sequence set forth in SEQ ID NO:34 and the second antigen-binding region comprises or consists of the sequence set forth in SEQ ID NO:17, wherein optionally X₄ is Y.
   In further preferred embodiments of the multispecific antibody of the invention,
(i) the first antigen-binding region comprises or consists of the sequence set forth in SEQ ID NO:1 and the second antigen-binding region comprises or consists of the sequence set forth in SEQ ID NO:36, or
(ii) the first antigen-binding region comprises or consists of the sequence set forth in SEQ ID NO:9 and the second antigen-binding region comprises or consists of the sequence set forth in SEQ ID NO:36, or
(iii) the first antigen-binding region comprises or consists of the sequence set forth in SEQ ID NO:25 and the second antigen-binding region comprises or consists of the sequence set forth in SEQ ID NO:36, or
(iv) the first antigen-binding region comprises or consists of the sequence set forth in SEQ ID NO:26 and the second antigen-binding region comprises or consists of the sequence set forth in SEQ ID NO:36, or
(v) the first antigen-binding region comprises or consists of the sequence set forth in SEQ ID NO:27 and the second antigen-binding region comprises or consists of the sequence set forth in SEQ ID NO:36, or
(vi) the first antigen-binding region comprises or consists of the sequence set forth in SEQ ID NO:28 and the second antigen-binding region comprises or consists of the sequence set forth in SEQ ID NO:36, or
(vii) the first antigen-binding region comprises or consists of the sequence set forth in SEQ ID NO:29 and the second antigen-binding region comprises or consists of the sequence set forth in SEQ ID NO:36, or
(viii) the first antigen-binding region comprises or consists of the sequence set forth in SEQ ID NO:30 and the second antigen-binding region comprises or consists of the sequence set forth in SEQ ID NO:36, or
(ix) the first antigen-binding region comprises or consists of the sequence set forth in SEQ ID NO:31 and the second antigen-binding region comprises or consists of the sequence set forth in SEQ ID NO:36, or
(x) the first antigen-binding region comprises or consists of the sequence set forth in SEQ ID NO:32 and the second antigen-binding region comprises or consists of the sequence set forth in SEQ ID NO:36, or
(xi) the first antigen-binding region comprises or consists of the sequence set forth in SEQ ID NO:33 and the second antigen-binding region comprises or consists of the sequence set forth in SEQ ID NO:36, or
(xii) the first antigen-binding region comprises or consists of the sequence set forth in SEQ ID NO:34 and the second antigen-binding region comprises or consists of the sequence set forth in SEQ ID NO:36.
   In further preferred embodiments of the multispecific antibody of the invention,
(i) the first antigen-binding region comprises or consists of the sequence set forth in SEQ ID NO:1 and the second antigen-binding region comprises or consists of the sequence set forth in SEQ ID NO:37, or
(ii) the first antigen-binding region comprises or consists of the sequence set forth in SEQ ID NO:9 and the second antigen-binding region comprises or consists of the sequence set forth in SEQ ID NO:37, or
(iii) the first antigen-binding region comprises or consists of the sequence set forth in SEQ ID NO:25 and the second antigen-binding region comprises or consists of the sequence set forth in SEQ ID NO:37, or
(iv) the first antigen-binding region comprises or consists of the sequence set forth in SEQ ID NO:26 and the second antigen-binding region comprises or consists of the sequence set forth in SEQ ID NO:37, or
(v) the first antigen-binding region comprises or consists of the sequence set forth in SEQ ID NO:27 and the second antigen-binding region comprises or consists of the sequence set forth in SEQ ID NO:37, or
(vi) the first antigen-binding region comprises or consists of the sequence set forth in SEQ ID NO:28 and the second antigen-binding region comprises or consists of the sequence set forth in SEQ ID NO:37, or
(vii) the first antigen-binding region comprises or consists of the sequence set forth in SEQ ID NO:29 and the second antigen-binding region comprises or consists of the sequence set forth in SEQ ID NO:37, or
(viii) the first antigen-binding region comprises or consists of the sequence set forth in SEQ ID NO:30 and the second antigen-binding region comprises or consists of the sequence set forth in SEQ ID NO:37, or
(ix) the first antigen-binding region comprises or consists of the sequence set forth in SEQ ID NO:31 and the second antigen-binding region comprises or consists of the sequence set forth in SEQ ID NO:37, or
(x) the first antigen-binding region comprises or consists of the sequence set forth in SEQ ID NO:32 and the second antigen-binding region comprises or consists of the sequence set forth in SEQ ID NO:37, or
(xi) the first antigen-binding region comprises or consists of the sequence set forth in SEQ ID NO:33 and the second antigen-binding region comprises or consists of the sequence set forth in SEQ ID NO:37, or
(xii) the first antigen-binding region comprises or consists of the sequence set forth in SEQ ID NO:34 and the second antigen-binding region comprises or consists of the sequence set forth in SEQ ID NO:37.
   In further preferred embodiments of the multispecific antibody of the invention,
(i) the first antigen-binding region comprises or consists of the sequence set forth in SEQ ID NO:1 and the second antigen-binding region comprises or consists of the sequence set forth in SEQ ID NO:38, or
(ii) the first antigen-binding region comprises or consists of the sequence set forth in SEQ ID NO:9 and the second antigen-binding region comprises or consists of the sequence set forth in SEQ ID NO:38, or
(iii) the first antigen-binding region comprises or consists of the sequence set forth in SEQ ID NO:25 and the second antigen-binding region comprises or consists of the sequence set forth in SEQ ID NO:38, or
(iv) the first antigen-binding region comprises or consists of the sequence set forth in SEQ ID NO:26 and the second antigen-binding region comprises or consists of the sequence set forth in SEQ ID NO:38, or
(v) the first antigen-binding region comprises or consists of the sequence set forth in SEQ ID NO:27 and the second antigen-binding region comprises or consists of the sequence set forth in SEQ ID NO:38, or
(vi) the first antigen-binding region comprises or consists of the sequence set forth in SEQ ID NO:28 and the second antigen-binding region comprises or consists of the sequence set forth in SEQ ID NO:38, or
(vii) the first antigen-binding region comprises or consists of the sequence set forth in SEQ ID NO:29 and the second antigen-binding region comprises or consists of the sequence set forth in SEQ ID NO:38, or
(viii) the first antigen-binding region comprises or consists of the sequence set forth in SEQ ID NO:30 and the second antigen-binding region comprises or consists of the sequence set forth in SEQ ID NO:38, or
(ix) the first antigen-binding region comprises or consists of the sequence set forth in SEQ ID NO:31 and the second antigen-binding region comprises or consists of the sequence set forth in SEQ ID NO:38, or
(x) the first antigen-binding region comprises or consists of the sequence set forth in SEQ ID NO:32 and the second antigen-binding region comprises or consists of the sequence set forth in SEQ ID NO:38, or
(xi) the first antigen-binding region comprises or consists of the sequence set forth in SEQ ID NO:33 and the second antigen-binding region comprises or consists of the sequence set forth in SEQ ID NO:38, or
(xii) the first antigen-binding region comprises or consists of the sequence set forth in SEQ ID NO:34 and the second antigen-binding region comprises or consists of the sequence set forth in SEQ ID NO:38.
   The first and second antigen-binding regions in the multispecific antibody may be arranged in various ways. In one embodiment, antigen-binding regions are connected to each other via a linker, such as a covalent linker. In one embodiment, the first antigen-binding region and the second antigen-binding region are covalently linked to each other via a peptide linker, e.g. a linker having a length of from 1 to 20 amino acids, e.g. from 1 to 10 amino acids, such as 2, 3, 4, 5, 6, 7, 8 or 10 amino acids. In one embodiment, the peptide linker comprises or consists of a sequence of 4 glycines following by a serine.

In some embodiments, the first antigen-binding region capable of binding human CD123 is located N-terminally of the second antigen-binding region capable of binding the human Vδ2 chain. In another embodiment, the first antigen-binding region capable of binding human CD123 is located C-terminally of the second antigen-binding region capable of binding the human Vδ2 chain.

Multispecific antibodies of the invention, such as bispecific antibodies, may contain further molecules, domains or polypeptide sequences beyond the first and second antigen-binding regions. In one embodiment, the multispecific antibody further comprises a half-life extension domain, i.e. a domain which prolongs the half-life of the molecules in the circulation of a human patient. In one embodiment, the multispecific antibody has a terminal half-life that is longer than about 168 hours when administered to a human subject. Most preferably the terminal half-life is 336 hours or longer. The “terminal half-life” of an antibody, when used herein refers to the time taken for the serum concentration of the polypeptide to be reduced by 50%, in vivo in the final phase of elimination.

In one embodiment, the multispecific antibody comprises an Fc region, preferably a human Fc region. In one embodiment, the multispecific antibody is in a VHH-Fc format, i.e. the antibody comprises two or more single-domain antigen-binding regions that are linked to each other via a human Fc region dimer, wherein each single-domain antigen-binding region is fused to an Fc region polypeptide (without CH1 or light chain sequences) and the two fusion polypeptides form a dimeric bispecific antibody via disulfide bridges in the hinge region.

Various method for making bispecific antibodies have been described in the art, e.g. reviewed by Brinkmann and Kontermann (2017) MAbs 9:182 and Labrijn et al (2019) Nature Reviews Drug Discovery 18: 585. In one embodiment of the present invention, the Fc region is a heterodimer comprising two Fc polypeptides, wherein the first antigen-binding region is fused to the first Fc polypeptide and the second antigen-binding region is fused to the second Fc polypeptide and wherein the first and second Fc polypeptides comprise asymmetric amino acid mutations that favor the formation of heterodimers over the formation of homodimers (see e.g. Ridgway et al. (1996) ‘Knobs-into-holes’ engineering of antibody CH3 domains for heavy chain heterodimerization. Protein Eng 9:617). In a further embodiment hereof, the CH3 regions of the Fc polypeptides comprise said asymmetric amino acid mutations, preferably the first Fc polypeptide comprises a T366W substitution and the second Fc polypeptide comprises T366S, L368A and Y407V substitutions, or vice versa, wherein the amino acid positions correspond to human IgG1 according to the EU numbering system. In a further embodiment, the cysteine residues at position 220 in the first and second Fc polypeptides have been deleted or substituted, wherein the amino acid position corresponds to human IgG1 according to the EU numbering system. In a further embodiment, the region comprises the sequence set forth in SEQ ID NO:35.

In some embodiments, the first and/or second Fc polypeptides contain mutations that render the Fc region inert, i.e. unable to mediate effector functions. In one embodiment, the first and second Fc polypeptides comprise a mutation at position 234 and/or 235, preferably the first and second Fc polypeptide comprise an L234F and an L235E substitution, wherein the amino acid positions correspond to human IgG1 according to the EU numbering system.

In a preferred embodiment,
the first antigen-binding region comprises the VH CDR1 sequence set forth in SEQ ID NO:2, the VH CDR2 sequence set forth in SEQ ID NO:3 and the VH CDR3 sequence set forth in SEQ ID NO:4 and the second antigen-binding region comprises the VH CDR1 sequence set forth in SEQ ID NO:18, the VH CDR2 sequence set forth in SEQ ID NO:19 and the VH CDR3 sequence set forth in SEQ ID NO:20, and
the first Fc polypeptide comprises the sequence set forth in SEQ ID NO:21 and the second Fc polypeptide comprises the sequence set forth in SEQ ID NO:22, or vice versa.
   In another preferred embodiment,
the first antigen-binding region comprises the VH CDR1 sequence set forth in SEQ ID NO:10, the VH CDR2 sequence set forth in SEQ ID NO:11 and the VH CDR3 sequence set forth in SEQ ID NO:12 and the second antigen-binding region comprises the VH CDR1 sequence set forth in SEQ ID NO:18, the VH CDR2 sequence set forth in SEQ ID NO:19 and the VH CDR3 sequence set forth in SEQ ID NO:20, and
the first Fc polypeptide comprises the sequence set forth in SEQ ID NO:21 and the second Fc polypeptide comprises the sequence set forth in SEQ ID NO:22, or vice versa.
   In a further preferred embodiment, the antibody of the invention consists of:
(i) a first polypeptide chain consisting of: a first antigen-binding region consisting of a sequence selected from the group consisting of the sequences set forth in SEQ ID NO:1, 9 and 25 to 34, the sequence set forth in SEQ ID NO:35, and the sequence set forth in SEQ ID NO:21, and
(i) a second polypeptide chain consisting of: a second antigen-binding region consisting of the sequence set forth in SEQ ID NO:17, wherein X₄ is Y, the sequence set forth in SEQ ID NO:35, and the sequence set forth in SEQ ID NO:22.
   In a further preferred embodiment, the antibody of the invention consists of:
(i) a first polypeptide chain consisting of: a first antigen-binding region consisting of a sequence selected from the group consisting of the sequences set forth in SEQ ID NO:1, 9 and 25 to 34, the sequence set forth in SEQ ID NO:35, and the sequence set forth in SEQ ID NO:22, and
(i) a second polypeptide chain consisting of: a second antigen-binding region consisting of the sequence set forth in SEQ ID NO:17, wherein X₄ is Y, the sequence set forth in SEQ ID NO:35, and the sequence set forth in SEQ ID NO:21.
   In one embodiment, the multispecific antibody of the invention is capable of mediating killing of CD123-expressing cells, such as C1R-neo cells or THP-1 cells, by Vγ9Vδ2 T cells.

Preferably, the antibody is capable of inducing killing C1R-neo cells through activation of Vγ9Vδ2 T cells with an EC50 value of 50 pM or less, such as 25 pM or less, e.g. 20 pM or less, such as 15 pM or less, e.g. 10 pM or less, or even 5 pM or less, such as 2 pM or less when tested as described in Example 5 herein.

In another embodiment, the antibody is capable of inducing killing THP-1 cells through activation of Vγ9Vδ2 T cells with an EC50 value of 100 pM or less, such as 50 pM or less, such as 25 pM or less, e.g. 20 pM or less, such as 15 pM or less, e.g. 10 pM or less, or even 5 pM or less, such as 2 pM or less when tested as described in Example 5 herein.

In a further embodiment, the multispecific antibody is capable of mediating killing of human patient-derived CD123-expressing bone-marrow-derived AML tumor cells. Such killing may e.g. be determined as described in Example 6 herein. In one embodiment, the multispecific antibody of the invention is capable of mediating specific cell death of more than 25%, such as more than 50%, at a concentration of 100 fM, as determined in the assay described in Example 6 herein.

In a further embodiment, the multispecific antibody is not capable of mediating killing of CD123-negative cells, such as CD123 negative human cells.

In a further embodiment, the multispecific antibody of the invention is capable of binding to the transiently CD123-expressing 293F cells with an EC50 of 50 nM or less, such as 20 nM or less, e.g. 10 nM or less, such as 5 nM or less, when tested as described in Example 3 herein.

In a further embodiment, the multispecific antibody of the invention is capable of binding to a recombinant CD123-Fc fusion protein with an EC50 of 50 nM or less, such as 20 nM or less, e.g. 10 nM or less, such as 5 nM or less, when tested as described in Example 4 herein.

In a further main aspect, the invention relates to an antibody comprising a first antigen-binding region capable of binding human CD123, wherein the first antigen-binding region is a single-domain antibody comprising:
(i) VH CDR1 sequence set forth in SEQ ID NO:2, the VH CDR2 sequence set forth in SEQ ID NO:3 and the VH CDR3 sequence set forth in SEQ ID NO:4, wherein preferably the first antigen-binding region comprises or consists of: a sequence selected from the group of sequences set forth in SEQ ID NO:1, 25 to 34, or a sequence having at least 90%, such as least 92%, e.g. at least 94%, such as at least 96%, e.g. at least 98% sequence identity to a sequence selected from the group of sequences set forth in SEQ ID NO:1, 25 to 34, or
(ii) the VH CDR1 sequence set forth in SEQ ID NO:10, the VH CDR2 sequence set forth in SEQ ID NO:11 and the VH CDR3 sequence set forth in SEQ ID NO:12, wherein preferably the first antigen-binding region comprises or consists of: the sequence set forth in SEQ ID NO:9, or a sequence having at least 90%, such as least 92%, e.g. at least 94%, such as at least 96%, e.g. at least 98% sequence identity to the sequence set forth in SEQ ID NO:9.

In one embodiment, in SEQ ID NO:2, X₁ is G. In another embodiment, X₁ is S.

In one embodiment, in SEQ ID NO:3, X₂ is A. In another embodiment, X₂ is T.

In one embodiment, in SEQ ID NO:4, X₃ is Y. In another embodiment, X₃ is F.

In one embodiment, X₁ is G, X₂ is A and X₃ is Y.

In another embodiment, X₁ is G, X₂ is A and X₃ is F.

In another embodiment, X₁ is G, X₂ is T and X₃ is Y.

In another embodiment, X₁ is G, X₂ is T and X₃ is F.

In one embodiment, X₁ is S, X₂ is A and X₃ is Y.

In another embodiment, X₁ is S, X₂ is A and X₃ is F.

In another embodiment, X₁ is S, X₂ is T and X₃ is Y.

In another embodiment, X₁ is S, X₂ is T and X₃ is F.

In a further main aspect, the invention relates to a pharmaceutical composition comprising an antibody, such as a multispecific antibody, according to the invention as described herein and a pharmaceutically-acceptable excipient.

Antibodies may be formulated with pharmaceutically-acceptable excipients in accordance with conventional techniques such as those disclosed in (Rowe et al., Handbook of Pharmaceutical Excipients, 2012 June, ISBN 9780857110275). The pharmaceutically-acceptable excipient as well as any other carriers, diluents or adjuvants should be suitable for the antibodies and the chosen mode of administration. Suitability for excipients and other components of pharmaceutical compositions is determined based on the lack of significant negative impact on the desired biological properties of the chosen antibody or pharmaceutical composition of the present invention (e.g., less than a substantial impact (10% or less relative inhibition, 5% or less relative inhibition, etc.) upon antigen binding).

A pharmaceutical composition may include diluents, fillers, salts, buffers, detergents (e.g., a nonionic detergent, such as Tween-20 or Tween-80), stabilizers (e.g., sugars or protein-free amino acids), preservatives, tissue fixatives, solubilizers, and/or other materials suitable for inclusion in a pharmaceutical composition. Further pharmaceutically-acceptable excipients include any and all suitable solvents, dispersion media, coatings, antibacterial and antifungal agents, isotonicity agents, antioxidants and absorption-delaying agents and the like that are physiologically compatible with an antibody of the present invention.

In a further main aspect, the invention relates to the multispecific antibody according to the invention as described herein for use as a medicament.

A multispecific antibody according to the invention enables creating a microenvironment that is beneficial for killing of tumor cells, in particular CD123-positive tumor cells, by Vγ9Vδ2 T cells.

Accordingly, in a further main aspect, the invention relates to the multispecific antibody according to the invention as described herein for use in the treatment of cancer. In a further main aspect, the invention relates to the multispecific antibody according to the invention as described herein for use in the treatment of acute myeloid leukemia, B-cell acute lymphoblastic leukemia, hairy cell leukemia, Hodgkin lymphoma, blastic plasmacytoid dendritic neoplasm, chronic myeloid leukemia, chronic lymphocytic leukemia, B-cell chronic lymphoproliferative disorders or myelodysplastic syndrome.

Similarly, the invention relates to a method of treating a disease comprising administration of a multispecific antibody according to the invention as described herein to a human subject in need thereof. In one embodiment, the disease is cancer, such as acute myeloid leukemia.

In some embodiments, the antibody is administered as monotherapy. However, antibodies of the present invention may also be administered in combination therapy, i.e., combined with other therapeutic agents relevant for the disease or condition to be treated.

“Treatment” or “treating” refers to the administration of an effective amount of an antibody according to the present invention with the purpose of easing, ameliorating, arresting, eradicating (curing) or preventing symptoms or disease states. An “effective amount” refers to an amount effective, at dosages and for periods of time necessary, to achieve a desired therapeutic result. An effective amount of a polypeptide, such as an antibody, may vary according to factors such as the disease stage, age, sex, and weight of the individual, and the ability of the antibody to elicit a desired response in the individual. An effective amount is also one in which any toxic or detrimental effects of the antibody are outweighed by the therapeutically beneficial effects. An exemplary, non-limiting range for an effective amount of an antibody of the present invention is about 0.1 μg/kg to 100 mg/kg, such as about 1 μg/kg to 50 mg/kg, for example about 0.01 to 20 mg/kg, such as about 0.1 to 10 mg/kg, for instance about 0.5, about 0.3, about 1, about 3, about 5, or about 8 mg/kg. Administration may be carried out by any suitable route, but will typically be parenteral, such as intravenous, intramuscular or subcutaneous.

Multispecific antibodies of the invention are typically produced recombinantly, i.e. by expression of nucleic acid constructs encoding the antibodies in suitable host cells, followed by purification of the produced recombinant antibody from the cell culture. Nucleic acid constructs can be produced by standard molecular biological techniques well-known in the art. The constructs are typically introduced into the host cell using an expression vector. Suitable nucleic acid constructs and expression vectors are known in the art. Host cells suitable for the recombinant expression of antibodies are well-known in the art, and include CHO, HEK-293, Expi293F, PER-C6, NS/0 and Sp2/0 cells.

Accordingly, in a further aspect, the invention relates to a nucleic acid construct encoding an antibody of the invention, such as multispecific antibody according to the invention. In one embodiment, the construct is a DNA construct. In another embodiment, the construct is an RNA construct.

In a further aspect, the invention relates to an expression vector comprising a nucleic acid construct encoding a multispecific antibody according to the invention.

In a further aspect, the invention relates to a host cell comprising one or more nucleic acid constructs encoding a multispecific antibody according to the invention or an expression vector comprising a nucleic acid construct encoding a multispecific antibody according to the invention.

TABLE 1
Sequence listing.

| SEQ | | | |
|---|---|---|---|
| ID. | code | Description | Sequence |
| 1 | 1D2 | VHH | EVQLVESGGGLVQAGGSLRLSCAASG |
| | | | RTASSYVMGWFRQAPGKEREFVAVIN |
| | | | WNGDSTYYTDSVKGRFAISRDNAKNT |
| | | | VYLQMNSLKPEDTAVYYCAADTRREW |
| | | | YRDGYWGPPARYEYDYRGQGTQVTVS |
| | | | S |
| 2 | 1D2 | CDR1 | GRTASSYVMX₁, |
| | | | wherein X₁ is G or S |
| 3 | 1D2 | CDR2 | VINWNGDSTYYX₂DSVKG, |
| | | | wherein X₂ is A or T |
| 4 | 1D2 | CDR3 | DTRREWYRDGX₃WGPPARYEYDY, |
| | | | wherein X₃ is |
| | | | Y or F |
| 5 | 1D2 | FR1 | EVQLVESGGGLVQAGGSLRLSCAA |
| | | | S |
| 6 | 1D2 | FR2 | WFRQAPGKEREFVA |
| 7 | 1D2 | FR3 | RFAISRDNAKNTVYLQMNSLKPED |
| | | | TAVYYCAA |
| 8 | 1D2 | FR4 | RGQGTQVTVSS |
| 9 | 1A3 | VHH | EVQLVESGGGLVQAGGSLRLSCAAS |
| | | | GRAINTYAMAWFRQAPGKERDFVAT |
| | | | ISYSGGTTDYAGSVKGRFTISRDNA |
| | | | ENTVYLQMNSLKPEDTAVYYCAARD |
| | | | RYNPLARNYNYWGQGTQVTVSS |
| 10 | 1A3 | CDR1 | GRAINTYAMA |
| 11 | 1A3 | CDR2 | TISYSGGTTDYAGSVKG |
| 12 | 1A3 | CDR3 | RDRYNPLARNYNY |
| 13 | 1A3 | FR1 | EVQLVESGGGLVQAGGSLRLSCAAS |
| 14 | 1A3 | FR2 | WFRQAPGKERDFVA |
| 15 | 1A3 | FR3 | RFTISRDNAENTVYLQMNSLKPED |
| | | | TAVYYCAA |
| 16 | 1A3 | FR4 | WGQGTQVTVSS |
| 17 | 5C8var | VHH | EVQLLESGGGSVQPGGSLRLSCAA |
| | | | SGRPFSNYAMSWFRQAPGKEREFV |
| | | | SAISWSGGSTSYADSVKGRFTISR |
| | | | DNSKNTLYLQMNSLRAEDTAVYYC |
| | | | AAQFSGADX₄GFGRLGIRGYEYDY |
| | | | WGQGTQVTVSS, |
| | | | wherein X₄ is Y, F or S |
| 18 | 5C8var | CDR1 | NYAMS |
| 19 | 5C8var | CDR2 | AISWSGGSTSYADSVKG |
| 20 | 5C8var | CDR3 | QFSGADX₄GFGRLGIRGYEYDY, |
| | | | wherein X₄ is Y, |
| | | | F or S |
| 21 | KiH | Fc | APEFEGGPSVFLFPPKPKDTLMISR |
| | (hole) | | TPEVTCVVVDVSHEDPEVKFNWYVD |
| | LFLE | | GVEVHNAKTKPREEQYNSTYRVVSV |
| | | | LTVLHQDWLNGKEYKCKVSNKALPA |
| | | | PIEKTISKAKGQPREPQVYTLPPSR |
| | | | DELTKNQVSLSCAVKGFYPSDIAVE |
| | | | WESNGQPENNYKTTPPVLDSDGSFF |
| | | | LVSKLTVDKSRWQQGNVFSCSVMHE |
| | | | ALHNHYTQKSLSLSPGK |
| 22 | KiH | Fc | APEFEGGPSVFLFPPKPKDTLMISR |
| | (knob) | | TPEVTCVVVDVSHEDPEVKFNWYVD |
| | LFLE | | GVEVHNAKTKPREEQYNSTYRVVSV |
| | | | LTVLHQDWLNGKEYKCKVSNKALPA |
| | | | PIEKTISKAKGQPREPQVYTLPPSR |
| | | | DELTKNQVSLWCLVKGFYPSDIAVE |
| | | | WESNGQPENNYKTTPPVLDSDGSFF |
| | | | LYSKLTVDKSRWQQGNVFSCSVMHE |
| | | | ALHNHYTQKSLSLSPGK |
| 23 | Human | | MVLLWLTLLLIALPCLLQTKEDPNP |
| | CD123 | | PITNLRMKAKAQQLTWDLNRNVTDI |
| | | | ECVKDADYSMPAVNNSYCQFGAISL |
| | | | CEVTNYTVRVANPPFSTWILFPENS |
| | | | GKPWAGAENLTCWIHDVDFLSCSWA |
| | | | VGPGAPADVQYDLYLNVANRRQQYE |
| | | | CLHYKTDAQGTRIGCRFDDISRLSS |
| | | | GSQSSHILVRGRSAAFGIPCTDKFV |
| | | | VFSQIEILTPPNMTAKCNKTHSFMH |
| | | | WKMRSHFNRKFRYELQIQKRMQPVI |
| | | | TEQVRDRTSFQLLNPGTYTVQIRAR |
| | | | ERVYEFLSAWSTPQRFECDQEEGAN |
| | | | TRAWRTSLLIALGTLLALVCVFVIC |
| | | | RRYLVMQRLFPRIPHMKDPIGDSFQ |
| | | | NDKLVVWEAGKAGLEECLVTEVQVV |
| | | | QKT |
| 24 | Human | | MVLAQGLLSMALLALCWERSLAGAE |
| | CD131 | | ETIPLQTLRCYNDYTSHITCRWADT |
| | | | QDAQRLVNVTLIRRVNEDLLEPVSC |
| | | | DLSDDMPWSACPHPRCVPRRCVIPC |
| | | | QSFVVTDVDYFSFQPDRPLGTRLTV |
| | | | TLTQHVQPPEPRDLQISTDQDHFLL |
| | | | TWSVALGSPQSHWLSPGDLEFEVVY |
| | | | KRLQDSWEDAAILLSNTSQATLGPE |
| | | | HLMPSSTYVARVRTRLAPGSRLSGR |
| | | | PSKWSPEVCWDSQPGDEAQPQNLEC |
| | | | FFDGAAVLSCSWEVRKEVASSVSFG |
| | | | LFYKPSPDAGEEECSPVLREGLGSL |
| | | | HTRHHCQIPVPDPATHGQYIVSVQP |
| | | | RRAEKHIKSSVNIQMAPPSLNVTKD |
| | | | GDSYSLRWETMKMRYEHIDHTFEIQ |
| | | | YRKDTATWKDSKTETLQNAHSMALP |
| | | | ALEPSTRYWARVRVRTSRTGYNGIW |
| | | | SEWSEARSWDTESVLPMWVLALIVI |
| | | | FLTIAVLLALRFCGIYGYRLRRKWE |
| | | | EKIPNPSKSHLFQNGSAELWPPGSM |
| | | | SAFTSGSPPHQGPWGSRFPELEGVF |
| | | | PVGFGDSEVSPLTIEDPKHVCDPPS |
| | | | GPDTTPAASDLPTEQPPSPQPGPPA |
| | | | ASHTPEKQASSFDFNGPYLGPPHSR |
| | | | SLPDILGQPEPPQEGGSQKSPPPGS |
| | | | LEYLCLPAGGQVQLVPLAQAMGPGQ |
| | | | AVEVERRPSQGAAGSPSLESGGGPA |
| | | | PPALGPRVGGQDQKDSPVAIPMSSG |
| | | | DTEDPGVASGYVSSADLVFTPNSGA |
| | | | SSVSLVPSLGLPSDQTPSLCPGLAS |
| | | | GPPGAPGPVKSGFEGYVELPPIEGR |
| | | | SPRSPRNNPVPPEAKSPVLNPGERP |
| | | | ADVSPTSPQPEGLLVLQQVGDYCFL |
| | | | PGLGPGPLSLRSKPSSPGPGPEIKN |
| | | | LDQAFQVKKPPGQAVPQVPVIQLFK |
| | | | ALKQQDYLSLPPWEVNKPGEVC |
| 25 | 1D2 | | EVQLVESGGGLVQPGGSLRLSCAAS |
| | var1 | | GRTASSYVMGWFRQAPGKEREFVSV |
| | | | INWNGDSTYYADSVKGRF |
| | | | TISRDNSKNTLYLQMNSLRAEDTAV |
| | | | YYCAADTRREWYRDGYWGPPARYEY |
| | | | DYRGQGTQVTVSS |
| 26 | 1D2 | | EVQLVESGGGLVQPGGSLRLSCAAS |
| | var2 | | GRTASSYVMGWVRQAPGKEREWVSV |
| | | | INWNGDSTYYADSVKGRFTISRDNS |
| | | | KNTLYLQMNSLRAEDTAVYYCAADT |
| | | | RREWYRDGYWGPPARYEYDYRGQGT |
| | | | LVTVSS |
| 27 | 1D2 | | EVQLVESGGGLVQPGGSLRLSCAAS |
| | var3 | | GRTASSYVMSWFRQAPGKEREWVAV |
| | | | INWNGDSTYYADSVKGRFTISRDNS |
| | | | KNTVYLQMNSLRAEDTAVYYCAADT |
| | | | RREWYRDGYWGPPARYEYDYRGQGT |
| | | | LVTVSS |
| 28 | 1D2 | | EVQLVESGGGVVQPGGSLRLSCAAS |
| | var4 | | GRTASSYVMSWFRQAPGKEREWVAV |
| | | | INWNGDSTYYADSVKGRFTISRDNS |
| | | | KNTLYLQMNSLRAEDTAVYYCAADT |
| | | | RREWYRDGYWGPPARYEYDYRGQGT |
| | | | TVTVSS |
| 29 | 1D2 | | EVQLVESGGGLVQPGGSLRLSCAAS |
| | var5 | | GRTASSYVMSWFRQAPGKEREFVAV |
| | | | INWNGDSTYYADSVKGRFTISRDNS |
| | | | KNTVYLQMNSLRAEDTAVYYCAADT |
| | | | RREWYRDGYWGPPARYEYDYRGQGT |
| | | | LVTVSS |
| 30 | 1D2 | | EVQLVESGGGLVQPGGSLRLSCAAS |
| | var6 | | GRTASSYVMGWVRQAPGKGLEWVSV |
| | | | INWNGDSTYYADSVKGRFTISRDNS |
| | | | KNTLYLQMNSLRAEDTAVYYCAADT |
| | | | RREWYRDGYWGPPARYEYDYRGQGT |
| | | | LVTVSS |
| 31 | 1D2 | | EVQLVESGGGLVQPGGSLRLSCAAS |
| | var7 | | GRTASSYVMSWVRQAPGKGLEWVSV |
| | | | INWNGDSTYYADSVKGRFTISRDNS |
| | | | KNTLYLQMNSLRAEDTAVYYCAADT |
| | | | RREWYRDGYWGPPARYEYDYRGQGT |
| | | | LVTVSS |
| 32 | 1D2 | | EVQLVESGGGLVQPGGSLRLSCAAS |
| | var8 | | GRTASSYVMSWFRQAPGKGLEWVAV |
| | | | INWNGDSTYYADSVKGR |
| | | | FTISRDNSKNTVYLQMNSLRAEDTA |
| | | | VYYCAADTRREWYRDGYWGPPARYE |
| | | | YDYRGQGTLVTVSS |
| 33 | 1D2 | | EVQLVESGGGLVQAGGSLRLSCAAS |
| | var9 | | GRTASSYVMGWFRQAPGKEREWVAV |
| | | | INWNGDSTYYTDSVKGRFAISRDNA |
| | | | KNTVYLQMNSLRAEDTAVYYCAADT |
| | | | RREWYRDGYWGPPARYEYDYRGQGT |
| | | | QVTVSS |
| 34 | 1D2 | | EVQLVESGGGLVQPGGSLRLSCAAS |
| | var10 | | GRTASSYVMGWFRQAPGKEREFVSV |
| | | | INWNGDSTYYADSVKGRFTISRDNS |
| | | | KNTLYLQMNSLRAEDTAVYYCAADT |
| | | | RREWYRDGFWGPPARYEYDYRGQGT |
| | | | QVTVSS |
| 35 | | Modified | AAASDKTHTCPPCP |
| | | hinge | |
| 36 | 6H4 | VHH | EVQLVESGGGLVQAGGSLRLSCAAS |
| | | | GRPFSNYGMGWFRQAPGKKREFVAG |
| | | | ISWSGGSTDYADSVKGRFTISRDNA |
| | | | KNTVYLQMNSLKPEDTAVYYCAAVF |
| | | | SGAETAYYPSDDYDYWGQGTQVTVS |
| | | | S |
| 37 | 6C1 | VHH | EVQLVESGGGLVQAGGSLRLSCAAS |
| | | | GRPFSNYGMGWFRQAPGKKRESVAG |
| | | | ISWSGGSTDYADSVKGRFTISRDNA |
| | | | KNTVYLQMNSLKPEDTAVYYCAAVF |
| | | | SGAETAYYPSDDYDYWGQGTQVTVS |
| | | | S |
| 38 | 5D3 | VHH | EVQLVESGGGLVQAGGSLRLSCAAS |
| | | | GRPFSNYAMGWFRQAPGKEREFVTV |
| | | | ISWSGGSTYYADSVKGRFTISRDNA |
| | | | KNTVYLQMNSLKPEDTAVYYCAAQF |
| | | | SGASTVVAGTALDYDYWGQGTRVTV |
| | | | SS |
| 39 | 6H4 | CDR1 | GRPFSNYGMG |
| 40 | 6H4 | CDR2 | GISWSGGSTDYADSVKG |
| 41 | 6H4 | CDR3 | VFSGAETAYYPSDDYDY |
| 42 | 6C1 | CDR1 | GRPFSNYGMG |
| 43 | 6C1 | CDR2 | GISWSGGSTDYADSVKG |
| 44 | 6C1 | CDR3 | VFSGAETAYYPSDDYDY |
| 45 | 5D3 | CDR1 | GRPFSNYAMG |
| 46 | 5D3 | CDR2 | VISWSGGSTYYADSVKG |
| 47 | 5D3 | CDR3 | QFSGASTVVAGTALDYDY |
| 48 | Vδ2 | | MQRISSLIHLSLFWAGVMSAIELVP |
| | | | EHQTVPVSIGVPATLRCSMKGEAIG |
| | | | NYYINWYRKTQGNTMTFIYREKDIY |
| | | | GPGFKDNFQGDIDIAKNLAVLKILA |
| | | | PSERDEGSYYCACDTLGMGGEYTDK |
| | | | LIFGKGTRVTVEPRSQPHTKPSVFV |
| | | | MKNGTNVACLVKEFYPKDIRINLVS |
| | | | SKKITEFDPAIVISPSGKYNAVKLG |
| | | | KYEDSNSVTCSVQHDNKTVHSTDFE |
| | | | VKTDSTDHVKPKETENTKQPSKSCH |
| | | | KPKAIVHTEKVNMMSLT |
| 49 | 5C8var1 | VHH-Fc | EVQLLESGGGSVQPGGSLRLSCAAS |
| | (Y105F) | | GRPFSNYAMSWFRQAPGKEREFVSA |
| | | | ISWSGGSTSYADSVKGRFTISRDNS |
| | | | KNTLYLQMNSLRAEDTAVYYCAAQF |
| | | | SGADFGFGRLGIRGYEYDYWGQGTQ |
| | | | VTVSSAAASDKTHTCPPCPAPEFEG |
| | | | GPSVFLFPPKPKDTLMISRTPEVTC |
| | | | VVVDVSHEDPEVKFNWYVDGVEVHN |
| | | | AKTKPREEQYNSTYRVVSVLTVLHQ |
| | | | DWLNGKEYKCKVSNKALPAPIEKTI |
| | | | SKAKGQPREPQVYTLPPSRDELTKN |
| | | | QVSLWCLVKGFYPSDIAVEWESNGQ |
| | | | PENNYKTTPPVLDSDGSFFLYSKLT |
| | | | VDKSRWQQGNVFSCSVMHEALHNHY |
| | | | TQKSLSLSPGK |
| 50 | 1D2 | VHH-Fc | EVQLVESGGGLVQAGGSLRLSCAAS |
| | | | GRTASSYVMGWFRQAPGKEREFVAV |
| | | | INWNGDSTYYTDSVKGRFAISRDNA |
| | | | KNTVYLQMNSLKPEDTAVYYCAADT |
| | | | RREWYRDGYWGPPARYEYDYRGQGT |
| | | | QVTVSSAAASDKTHTCPPCPAPEFE |
| | | | GGPSVFLFPPKPKDTLMISRTPEVT |
| | | | CVVVDVSHEDPEVKFNWYVDGVEVH |
| | | | NAKTKP |
| | | | REEQYNSTYRVVSVLTVLHQDWLNG |
| | | | KEYKCKVSNKALPAPIEKTISKAKG |
| | | | QPREPQVYTLPPSRDELTKNQVSLS |
| | | | CAVKGFYPSDIAVEWESNGQPENNY |
| | | | KTTPPVLDSDGSFFLVSKLTVDKSR |
| | | | WQQGNVFSCSVMHEALHNHYTQKSL |
| | | | SLSPGK |
| 51 | 7A5 | Heavy chain | QVQLQQSGAELARPGASVKLSCKAS |
| | | | GFTFTDHYINWVKQRTGQGLEWIGQ |
| | | | IYPGNGNTYYNEKFKGKATLTADKS |
| | | | SSTAYMQLSSLTSEDSAVYFCAPNY |
| | | | GDYTLDFWGQGTSVTVSSASTKGPS |
| | | | VFPLAPSSKSTSGGTAALGCLVKDY |
| | | | FPEPVTVSWNSGALTSGVHTFPAVL |
| | | | QSSGLYSLSSVVTVPSSSLGTQTYI |
| | | | CNVNHKPSNTKVDKKVEPKSCDKTH |
| | | | TCPPCPAPEFEGGPSVFLFPPKPKD |
| | | | TLMISRTPEVTCVVVDVSHEDPEVK |
| | | | FNWYVDGVEVHNAKTKPREEQYNST |
| | | | YRVVSVLTVLHQDWLNGKEYKCKVS |
| | | | NKALPAPIEKTISKAKGQPREPQVY |
| | | | TLPPSRDELTKNQVSLWCLVKGFYP |
| | | | SDIAVEWESNGQPENNYKTTPPVLD |
| | | | SDGSFFLYSKLTVDKSRWQQGNVFS |
| | | | CSVMHEALHNHYTQKSLSLSPGKAA |
| | | | AEPEA |
| 52 | 7A5 | Light chain | DIVMSQSPSSLAVSVGEKVTMSCKS |
| | | | SQSLLYSSNQKNYLAWYQQKPGQSP |
| | | | KLLIYWASTRESGVPDRFTGSGSGT |
| | | | DFTLTISSVKAEDLAVYYCQQYYRY |
| | | | HTFGTGTKLEIKRTVAAPSVFIFPP |
| | | | SDEQLKSGTASVVCLLNNFYPREAK |
| | | | VQWKVDNALQSGNSQESVTEQDSKD |
| | | | STYSLSSTLTLSKADYEKHKVYACE |
| | | | VTHQGLSSPVTKSFNRGEC |
| 53 | C-tag | | AAAEPEA |

All references, articles, publications, patents, patent publications, and patent applications cited herein are incorporated by reference in their entireties for all purposes. However, mention of any reference, article, publication, patent, patent publication, and patent application herein is not, and should not be, taken as acknowledgment or any form of suggestion that they constitute valid prior art or form part of the common general knowledge in any country in the world.

### EXAMPLES

### Example 1: Selection and Identification of Anti-CD123 VHH from Phage Display Libraries Made from Animals Immunized with C1R-CD1d Cells

Llama Glama (2 animals) were immunized with CD123 expressing C1R-CD1d cells, after which ‘immune’ VHH phage antibody libraries were made as described (Lameris et al., 2016 Immunology 149:111). These libraries were used for phage selections on captured recombinant human CD123 or directly coated CD123 antigen (extra-cellular domain, Sino Biological). A single, or two consecutive rounds of selection were performed. After one and two rounds of phage selection, single phage clones were screened for binding in ELISA to the recombinant captured antigen. Those clones that scored positive for binding were sequenced and all clones having a different sequence were then tested for binding to the cell line used for immunization in flow cytometry. The VHH clones showing binding in FACS were then selected for further characterization. Eight different clones were identified and termed: 1E2, 1B4, 1A3, 2D11, 1D2, 1E4, 1H1 and 1F1.

### Example 2: Synthetic Gene Synthesis, Production and Purification of Bispecific VHH

The sequences of CD123-specific VHH domain antibodies were then re-formatted to bispecific VHH with a Vδ2 specific VHH (5C8var1; SEQ ID NO:17, wherein X₄ is Y) in the orientation: N-term-anti-CD123 VHH-linker-anti-Vδ2 VHH-C-tag. The Vδ2 specific VHH used is set forth in SEQ ID NO:17 (wherein X₄ is Y). The linker between the two VHH domains was a glycine(G)-serine(S) stretch with the sequence G4S. The cDNAs encoding these proteins were made by synthetic gene synthesis at Genscript and then cloned into the eukaryotic expression vector pCDNA3.1+ (Thermofisher Scientific) by directional cloning. The proteins were expressed by transient transfection in Hek293E cells and then (after 5 days of expression) purified from the conditioned cell culture supernatant using Capture Select C-tag affinity matrix (Thermo Fisher Scientific) according to the supplier's protocol. Purified bispecific VHH was always >95% pure as determined by SDS-PAGE analysis using Coomassie staining and contained very low levels of endotoxin (<0.5 EU/mg).

### Example 3: Specificity of Binding of Bispecific Anti-CD123 x Vδ2 VHH in ELISA

Recombinant, purified CD123 antigen (extra-cellular domain; Sino Biological), or an Fc-fusion of the CD123 antigen (Bio-Techne/R&D Systems) was coated to the wells of an ELISA plate (Greiner) in PBS at a concentration of 2 μg/ml. As a negative control, wells were coated with 1% (w/v) BSA. An in-house designed, produced and purified recombinant form of the extra-cellular domains of the human Vγ9 and Vδ2 TCR chains fused to a human Fc was also coated as antigen at 2 μg/ml. After coating and blocking of the wells with 2% (w/v) BSA, bispecific VHH proteins were tested for binding at a saturating concentration of 50 nM and bound VHH was detected using an HRP-labeled anti-VHH antibody (Genscript) and staining using 3,3′,5,5′-tetramethylbenzidine (TMB)/H2O2.

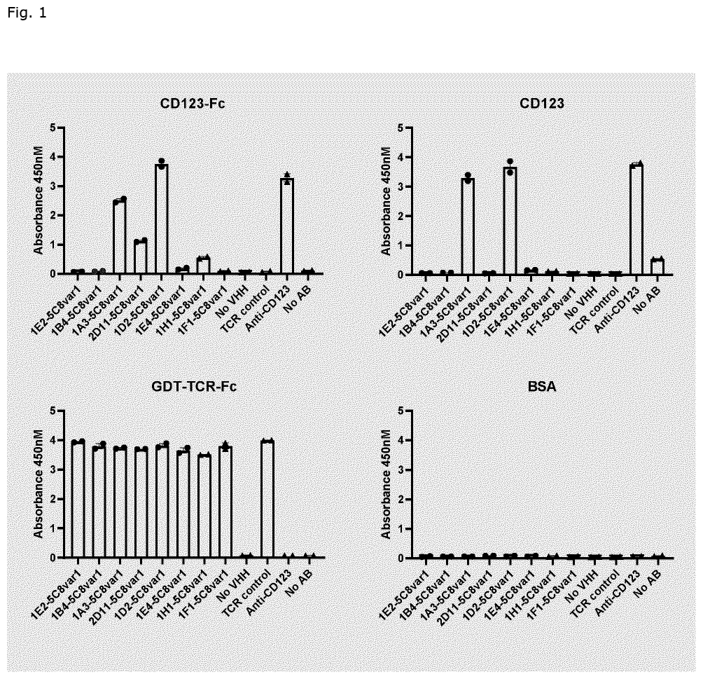

FIG. 1 shows that in ELISA, only 1A3-5C8var1 and 1D2-5C8var1 showed strong and specific binding to both CD123 and the γδ-TCR (gamma-delta T cell receptor). The other bispecifics bound weakly or did not bind CD123.

### Example 4: Specificity of Binding of Bispecific Anti-CD123 x Vδ2 VHH using Flow Cytometry

Expression constructs for human CD123 and for the common β chain of the receptor (CD131) were purchased from Invivogen. Plasmids were transformed to chemically competent DH5α bacteria and a single colony growing on selective medium was used to inoculate a 50 ml culture to amplify both constructs. Purified DNA was then transfected to freestyle 293F cells using polyethylene imine (PEI). Either plasmid alone, or a mix of the two plasmids was used for transfection. As negative control, untransfected cells were also used for flow cytometry. A day after transfection, cells were used to test the binding of the bispecific VHH using staining in FACS. Briefly, binding of a saturating concentration of 100 nM bispecific VHH to transfected cells was detected with an AF647-labeled anti-VHH (Genscript) antibody and staining was visualized using a FACS Celesta (Becton and Dickinson).

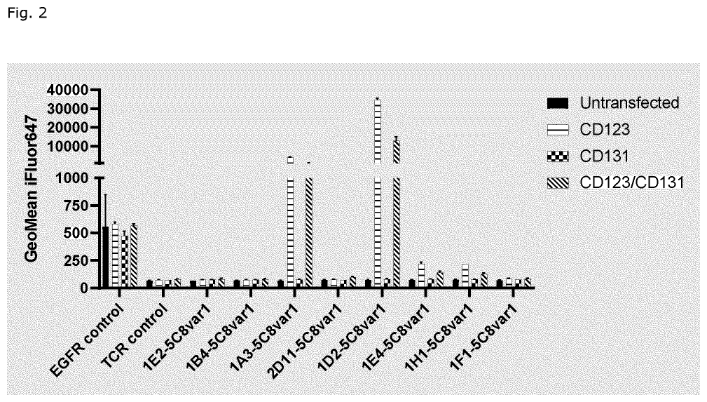

FIG. 2 shows that both 1A3-5C8var1 and 1D2-5C8var1 strongly and specifically recognized CD123, when the antigen was expressed alone, or when expressed in conjunction with CD131. 1D2-5C8var1 gave the strongest signals in flow cytometry.

To determine the apparent affinity of 1D2-5C8var1 for CD123 using flow cytometry, a concentration range of this bispecific VHH was tested for binding to transiently transfected 293F cells expressing either CD123, CD131 or both CD123 and -131. The former cells (expressing CD131) were used as negative control.

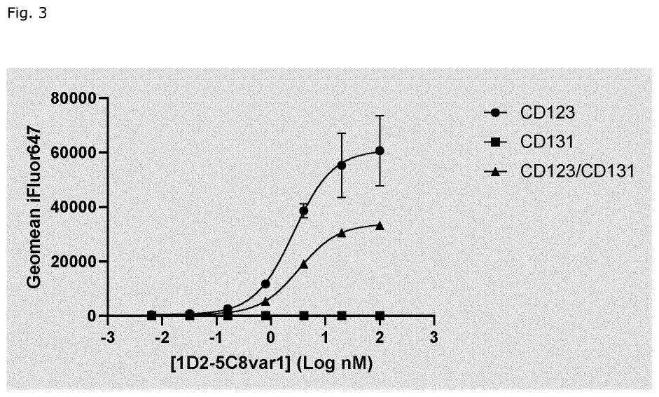

FIG. 3 shows again that 1D2-5C8var1 was exquisitely specific for CD123, as only cells (transiently) expressing CD123, but not CD131 were recognized. In addition, data show that the apparent affinity of 1D2-5C8var1 binding to CD123 as determined using flow cytometry was approximately 3 nM. This value was comparable for binding to CD123 alone, or to co-expressed CD123 and -131.

### Example 5: Affinity Determination of 1D2-5C8var1 for Binding CD123 Using Biolayer Interferometry (BLI)

To determine the kinetics of binding of 1D2-5C8var1 to CD123, recombinant purified CD123-Fc fusion protein (Bio-Techne/R&D Systems) was loaded to a density of 1nm onto anti-human IgG Fc Capture sensors (using a concentration of 5 μg/ml) for an Octet Red96e (Sartorius) instrument. Different sensors were then dipped in different concentrations of 1D2-5C8var1; dilutions were made in 10× kinetic buffer (10×KB) provided by the supplier. From the obtained sensorgrams, the kinetic association- and dissociation rate constants were determined by curve fitting.

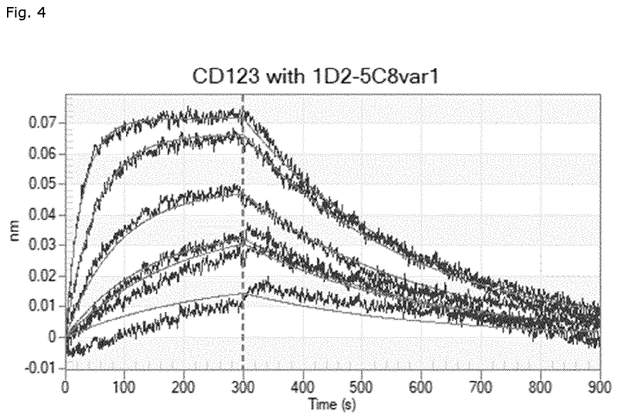

FIG. 4 shows the actual sensorgrams that were used for curve fitting. The latter is depicted as straight lines in the figure. This was used to determine the kinetic association- and dissociation rate constants and thereby the affinity of the anti-CD123 VHH 1D2. Measurements were performed twice and the affinity of 1D2-5C8var1 for CD123 was measured to be between 3 and 5 nM.

### Example 6: CD123-Dependent, 1D2-5C8var1 Mediated Vγ9Vδ2 T Cell Activation and T Cell Mediated Target Cell Cytotoxicity

Buffy coats were obtained from Sanquin (Amsterdam, the Netherlands). PBMC were isolated from these buffy coats by Ficoll density gradient centrifugation using described procedures. Highly pure Vγ9Vδ2 T cells were obtained by MACS using a Vδ2 specific antibody and these were expanded using published methods (de Bruin et al., 2016 Clin Immunol. 169:128). The CD123-positive B lymphoblast, EBV-transformed C1R neo cell line (CRL-2369) and CD123-positive AML-derived THP-1 cell line (TIB-202) were obtained from the American type culture collection and cultured according to the supplier's instructions. Target cells were labelled with cell trace violet (CTV) for 20 minutes at 37° C. To measure T cell activation, cells were stained for the activation marker CD107a (or LAMP-1, lysosomal associated membrane protein-1) that becomes cell surface-exposed once cells degranulate. A concentration range of bispecific antibody was incubated for 4 hours with a 1:1 mix of target cells and expanded Vγ9Vδ2 T cells (50,000 cells each) in a final volume of 100 μl in the presence of a PE-labeled anti-CD107A antibody. After incubation, cells were washed and stained with a mix of fluorescently-labeled anti-CD3 and anti-Vγ9 antibodies to identify the T cells and a live/dead stain (7-AAD). Samples were analyzed using a FACS Celesta (Beckton and Dickinson). To assess T-cell mediated target cell cytotoxicity, essentially the same setup was used, only no anti-CD107A antibody was added and CTV-labeled target cells and Vγ9Vδ2 T cells were incubated for 24 hours in the presence of the bispecific VHH. Cells were stained again for CD3 and Vγ9 at the end of the assay and analyzed using flow cytometry in the presence of a live/dead stain (7-AAD).

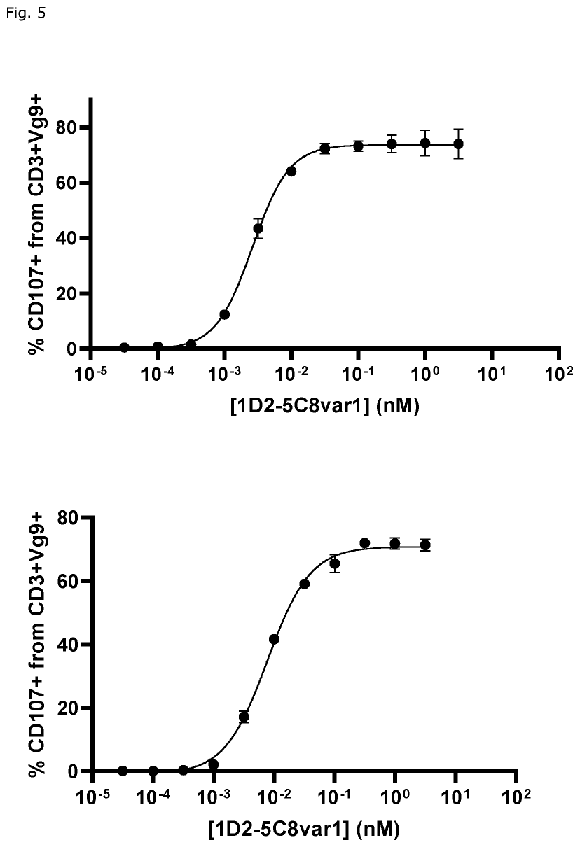

FIG. 5 shows that 1D2-5C8var1 caused potent T cell activation with an EC50 in the pM range. In the absence of target cells, a high concentration of 1D2-5C8var1 caused only background activation (data not shown). The potency of 1D2-5C8var1 to induce T cell activation was slightly dependent on the T cell donor used; EC50 values ranged between 3 and 13 pM.

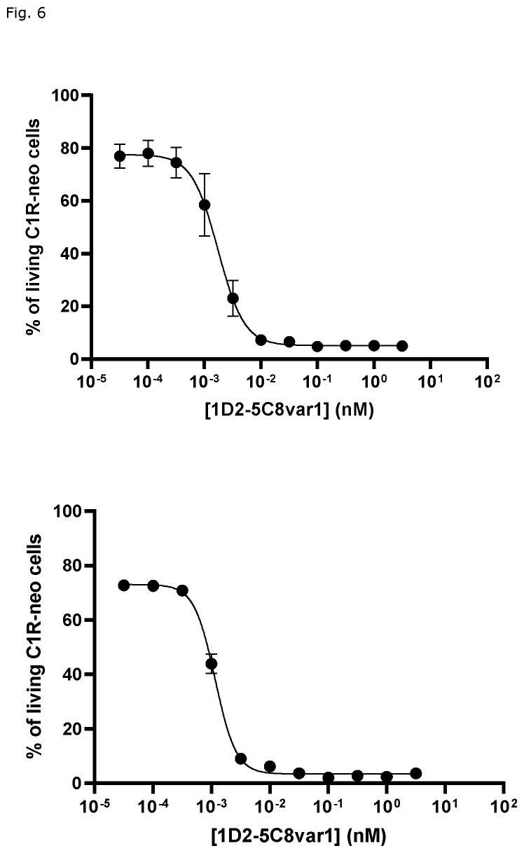

To determine whether the observed T cell activation would also result in target cell lysis, a cytotoxicity assay was performed using 1:1 effector to target (E:T) ratio and a 24 hour timepoint. FIG. 6 shows that 1D2-5C8var1 potently induced C1R-neo target cell lysis in the presence of expanded Vγ9Vδ2 T cells. The EC50 for cytotoxicity was determined by curve fitting to be between 1 and 2 pM, dependent on the T cell donor used (data for two donors are depicted).

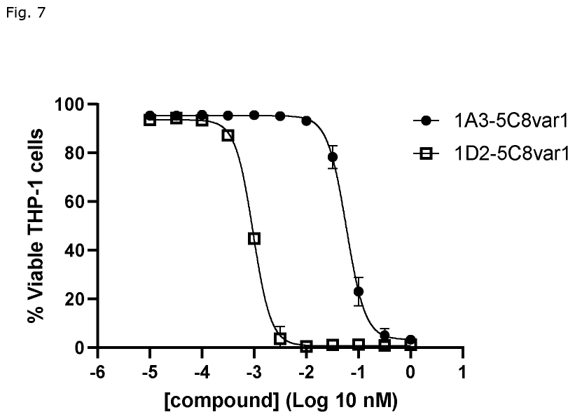

The same cytotoxicity assay was repeated with a CD123-positive AML cell line: THP-1. The potency of 1D2-5C8var1 to induce THP-1 target cell lysis was very comparable: EC50 was measured to be 1 μM. In contrast: the potency of 1A3-5C8var1 to induce target cell lysis was measured to be around 50 pM: FIG. 7.

### Example 7: Bispecific VHH Mediated-, Vγ91/62 T Cell Activation and T Cell Induced Lysis of Primary AML Cells

25,000 bone marrow-derived mononuclear cells from an AML patient were co-cultured overnight at a 1:1 ratio with expanded Vγ9Vδ2 T cells derived from a healthy donor. The cells were cultured in the presence of a PE-labeled CD107a antibody and a concentration range of 1D2-5C8var1 (ranging from 10 fM to 100 nM). The cells were harvested, washed and labeled for 30″ at 4° C. with an antibody mix containing fluorescently-labeled anti-CD45, CD117, CD34, CD33 and CD2 antibodies. After washing, the cells were resuspended in a mixture of live/dead stain (7AAD) and 123 counting beads, and subsequently analyzed using an LSRFortessa flow cytometer.

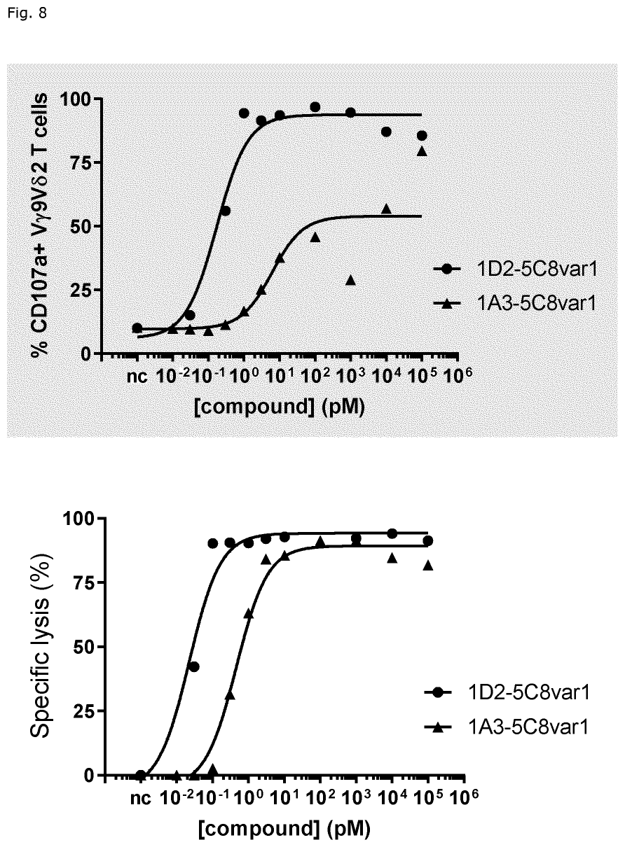

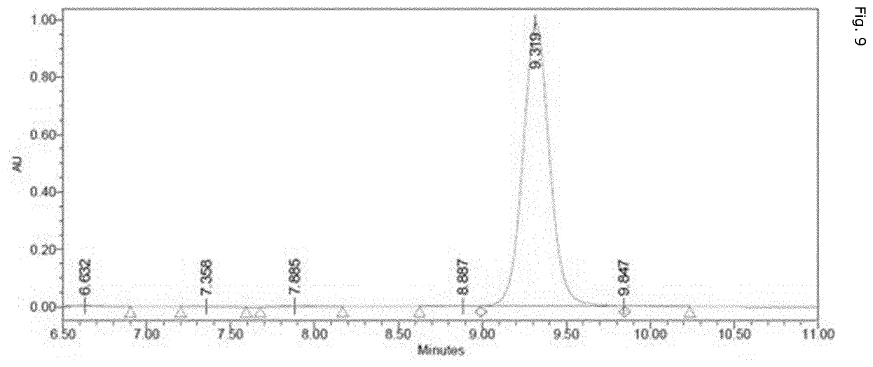

FIG. 8 shows that both bispecific VHH compounds were capable of inducing potent T cell activation dependent on CD123 positive primary AML blasts. In addition, the compounds caused a high level of tumour cell lysis and both showed a significant potency in this cytotoxicity. The EC50 values could not unequivocally be determined from the obtained curves, but were in the fM range (below 1 pM).

### Example 8: Humanization of Anti-CD123 VHH 1D2 Using CDR Grafting

The 1D2 VHH antibody fragment was humanized using CDR-grafting technology (see e.g. U.S. Pat. No. 5,225,539 and Williams, D. G. et al., 2010, Antibody Engineering, volume 1, Chapter 21). First, human germline sequences were identified using IgBLAST (Ye J. et al., 2013, Nucleic Acids Res. 41:W34-40). As closest human germline sequence, V-gene IGVH3-23*04 was identified (78.4% identity). This germline sequence was used to directly graft the llama CDRs (91.8% identity with human germline IGVH3-23*04), resulting in the following cDNA construct: SEQ ID NO: 31 Next, the NCBI NR database (downloaded Sep. 27, 2020) was queried using BLASTP (version 2.10.0+) to identify human template sequences that demonstrated the highest identity to the 1D2 sequence. Two VH sequences were identified that demonstrated a similarity score of 70% or higher and that displayed similar CDR lengths, preferably identical to those in 1D2 CDR1, CDR2, CDR3, respectively. The frameworks encoded by GenBank (Benson, D. A. et al., 2013, Nucleic Acids Res. 41(D1):D36-42) accession #CAD60357.1, and AKU38567.1 were selected as templates for grafting of the 1D2 CDRs, resulting in the following cDNA constructs: SEQ ID NO: 28 and 32, respectively. Framework and CDR definition were those as determined by Kabat et al. (“Sequences of Proteins of Immunological Interest”, Kabat, E., et al., US Department of Health and Human Services, (1983)). To understand the effect of humanized framework residues on the structure of the VHH, a homology model of 1D2 VHH was made using the ‘Antibody Prediction’-tool (default parameters) within BioLuminate 4.2.156 (Schrodinger). The homology model was built on basis of PDB ID 6GKU. The CDRs were grafted in silico to study the effect of the human residues for features as loop conformation of the CDRs, the hydrophobicity of the surface, and structural integrity (e.g. increased rigidity). The resulting constructs were checked for these features, resulting in the design of additional constructs: SEQ ID NO: 25, 26, 27, 29, 30, 33 and 34. The sequences of these humanized 1D2-VHHs were then re-formatted to bispecific VHH with a Vδ2 specific VHH (5C8var1; SEQ ID NO:17, wherein X₄ is Y) in the orientation: N-term-humanized-anti-1D2 VHH-linker-anti-Vδ2 VHH-C-tag. The cDNAs encoding these molecules were then synthesized and cloned into an expression vector for the expression in HEK293E cells. Protein was made by transient transfection of the cells and was purified from the culture supernatant using C-tag affinity chromatography, followed by preparative size exclusion chromatography.

### Example 9: Affinity Determination of Humanized 1D2-5C8var1(Y105F) Variants for Binding CD123 Using Biolayer Interferometry (BLI)

To determine the kinetics of binding of humanized 1D2-5C8var1 variants to CD123, recombinant purified CD123-Fc fusion protein (Bio-Techne/R&D Systems) was loaded to a density of 1 nm onto anti-human IgG Fc Capture sensors (using a concentration of 5 μg/ml) for an Octet Red96e (Sartorius) instrument. Different sensors were then dipped in different concentrations of humanized 1D2-5C8var1 variants, starting at 50 nM and two-fold dilutions thereof; dilutions were made in 10× kinetic buffer (10×KB) provided by the supplier. From the obtained sensorgrams, the kinetic association- and dissociation rate constants were determined by curve fitting. The association- and dissociation rate constants, when fitting was possible, were used to calculate the affinity of the humanized 1D2-5C8var1 variants for binding to CD123. Measurements were performed twice and the affinity of the different humanized 1D2-5C8var1 variants for CD123 ranged from 2.6 nM, similar compared to parental 1D2, to non-binding variants as shown in Table 2. For reference, the non-humanized (parental) 1D2 was included in these experiments.

TABLE 2
affinity of humanized variants of 1D2 to recombinant CD123

| SEQ ID No: | KD (nM) | SD |
|---|---|---|
| 1 | 3.1 | 0.7 |
| 25 | 7.2 | 0.4 |
| 26 | 24.5* | |
| 27 | 14.9 | 4.3 |
| 28 | 20.0 | 7.5 |
| 29 | 7.7 | 1.5 |
| 30 | 29.1* | |
| 31 | n.b. | |
| 32 | 44.1* | |
| 33 | 2.6 | 0.5 |
| 34 | 10.4 | 7.5 |

*low binding (>20 nM), one measurement only, n.b.: no binding observed

### Example 10: Half-Life Extended (Fc Containing) Bispecific Constructs

The 1D2-5C8var1 bispecific VHH was re-formatted into a therapeutic antibody format containing a human Fc domain. Both VHH domains were coupled to a human IgG1 Fc (i.e. CH2 and CH3) domain with the following characteristics: the VHH was coupled to a modified hinge (AAA, followed by SDKTHTCPPCP) and human CH2 and CH3 domains. The CH2 domain was Fc-silenced by the LFLE mutational pair (L234F, L235E) and the CH3 domains were mutated with the ‘knobs-into-holes’ mutations (knob: T366W and hole: T366S, L368A and Y407V) that enforce hetero-dimerization, upon co-expression of the two chains in the same cell. This mutational pair has been described in the scientific literature (Ridgway et al. (1996) Protein Eng 9:617). The C-terminus of the anti-Vγ9Vδ2 heavy chain was equipped with a C-terminal tag for purification purposes (AAAEPEA (SEQ ID NO:53)). The sequences of the constructs are set forth in SEQ ID NO:49 and SEQ ID NO:50. The resulting antibody construct was termed 1D2-5C8var1(Y105F)-Fc.

Protein was made via co-transfection of the encoding two expression vectors in HEK293E cells and purification from the culture supernatant by means of C-tag affinity chromatography, followed by preparative size exclusion chromatography. This yielded a highly monomeric protein preparation of 1D2-5C8var1(Y105F)-Fc: FIG. 9.

### Example 11: 1D2-5C8var1(Y105F)-Fc Induces Target-Dependent T Cell Activation and Causes T Cell-Mediated Target Cell Cytotoxicity with an Equal Potency as that of the Bispecific VHH

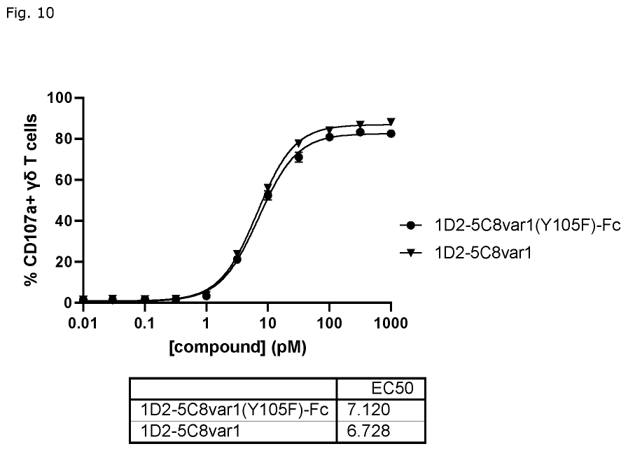

1D2-5C8var1(Y105F)-Fc was then tested for its capacity to induce target-dependent Vγ9Vδ2 T cell activation in a co-culture of Vγ9Vδ2 T cells and THP-1 tumor cells (in a 1:1 ratio). Vγ9Vδ2 T cells were expanded from the blood of a healthy donor using procedures known in the art. The THP-1 cell line (ATCC cat. Nr. TIB-202) was cultured as recommended by the supplier. In a 4 hours co-culture of both cell types, the activation of the Vγ9Vδ2 T cells was measured by staining for CD107a and measuring the percentage of CD107a positive cells by flow cytometry. FIG. 10 shows that 1D2-5C8var1(Y105F)-Fc induced target-dependent T cell activation (no activation was observed in a co-culture of Vγ9Vδ2 T cells and tumor cells in the absence of compound; data not shown). The EC50 was typically in the pM range, ranging from 4 to 16 pM (dependent on the donor used).

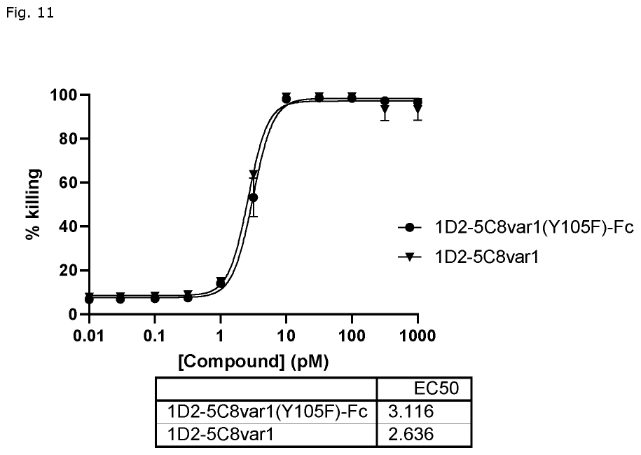

Remarkably, the potency of the Fc-containing molecule in inducing Vγ9Vδ2 T cell activation was not measurably different from that of the bispecific VHH. This was observed for three different independent T cell donors. To determine whether this T cell activation also resulted in target cell lysis, the viability of the THP-1 target cells was measured after 24 hours of co-culture (1:1 ratio) with Vγ9Vδ2 T cells in the presence of antibody. Vγ9Vδ2 T cells were isolated from the blood of healthy donors and expanded using standardized protocols. The day before the assay, the THP-1 target cell line was labeled with cell trace violet (CTV) to be able to distinguish it from the effector cell population in flow cytometry. After 24 hours of co-culture in the presence of increasing concentrations of compound, the percentage of living target cells was determined: FIG. 11.

FIG. 11 shows that the bispecific VHH and Fc-containing counterpart both induced strong T-cell mediated target cell cytotoxicity and that the potency of both molecules in causing target cell lysis was not measurably different. EC50 values ranged between 1 and 3 pM, dependent on the donor used. No target cell lysis was observed in the co-culture in the absence of compound (data not shown). After 24 hours, all target cells in the assay were killed.

### Example 12: 1D2-5C8var1(Y105F)-Fc Causes Preferential Kill of Tumor Cells Over Target-Positive Normal Cells

To determine to what extent the antibody induced killing of CD123-positive normal cells, plasmacytoid dendritic cells (pDCs) that are known to express CD123 (Collin et al., 2013 Immunology 140, 1:22-30) were enriched from the peripheral blood mononuclear cell (PBMC) fraction isolated from the blood from two healthy donors using MACS sorting (Miltenyi Biotech, Cat. Nr. 130-097-415). The THP-1 cell line was used as tumor cell line expressing CD123. Using staining for CD123 and analysis by flow cytometry, it was shown that the expression level of CD123 was approximately ten-fold higher on pDCs than on the THP-1 cell line: FIG. 12.

The cytotoxic effects of the CD123 targeting compound in combination with Vγ9Vδ2 T cells on a mix of target cells were then determined in a co-culture of the THP-1 cell line, pDCs and Vγ9Vδ2 T cells in the ratio 1:1:2. Vγ9Vδ2 T cells were isolated from the blood of healthy donors and expanded using standardized protocols. The day before the assay, the THP-1 target cell line was labeled with cell trace violet (CTV) to be able to distinguish it from the effector cell population and other target cells in flow cytometry. After 24 hours of co-culture in the presence of increasing concentrations of compound, the percentage of living target cells was determined by staining for Vγ9 (T cells), CD303 (pDC), CTV (THP-1) and CD123 (THP-1 and pDC) and analysed by flow cytometry.

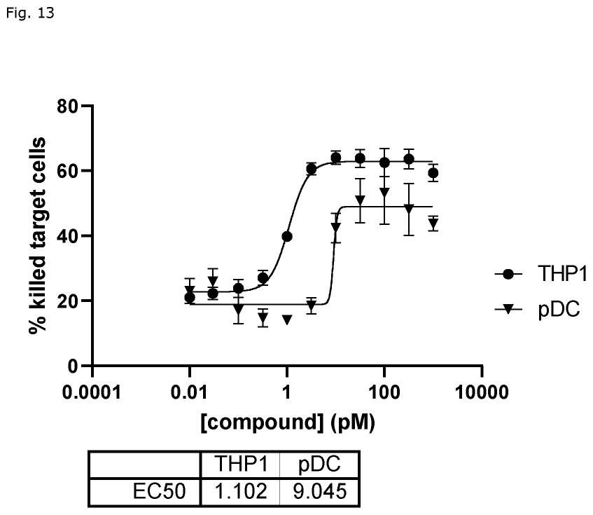

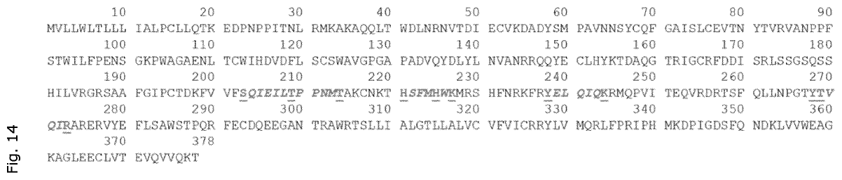

FIG. 13 shows that the bispecific antibody induced the expected THP-1 target cell lysis (FIG. 11) with a potency (EC50) that was around 1 pM. Remarkably however, despite the ten-fold higher expression level of the CD123 target molecule on pDCs (FIG. 12), these cells were far less affected. The maximal lysis observed was lower and the EC50 found in the assay was almost 10-fold higher than that found for THP-1 cell lysis. Results for donor nr. 2 were similar (EC50 values of 1 and 11 pM for THP-1 and pDCs respectively; data not shown). These data show a preferential induction of lysis of tumor cells over target-positive normal cells by the compound and Vγ9Vδ2 T cells.

### Example 13: Epitope Mapping Reveals the 1D2 VHH to Bind to a Membrane-Proximal Epitope

To determine what epitope on CD123 was recognized by the anti-CD123 VHH 1D2, this epitope was mapped using a mass-spectrometry based method (Pimenova et al., 2008 J Mass Spectrom. 43(2): 185-95). A number of residues in the CD123 molecule were found cross-linked to the antibody: FIG. 14.

FIG. 14 identifies the epitope of 1D2 to be present in the region of amino acid 203-273 of human CD123. When these residues are highlighted in the crystal structure of the molecule (PDB ID 5UV8: Broughton et al., 2018 Nat Commun. 9: 386), this maps to the second domain that is most membrane proximal and covers a surface area of 1011 Å².

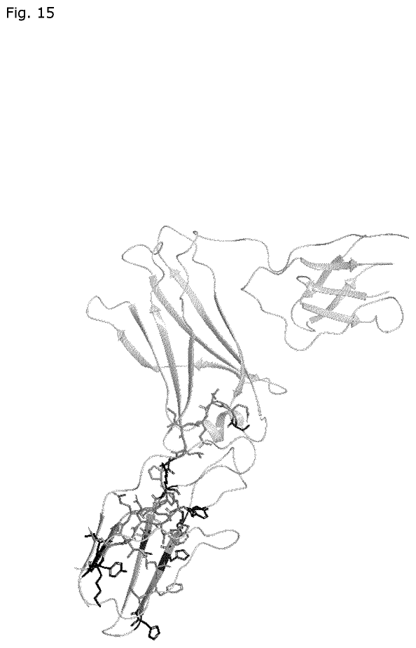

FIG. 15 shows that the epitope recognized by the lead anti-CD123 antibody is located close to the membrane. The distance spanned is about 40 Å, which is not uncommon for an epitope. All CDR regions of the 1D2 VHH were found cross-linked to the antigen, with a particularly strong signal for the CDR3.

A similar experiment was performed to determine what epitope on CD123 was recognized by the anti-CD123 VHH 1A3. Residues H225, H231, R234, T251, R255 and T267 of CD123 were found to be cross-linked to the 1A3 antibody.

### Example 14: 1D2x5C8var1(Y105F)-Fc Shows a Favorable Stability Profile

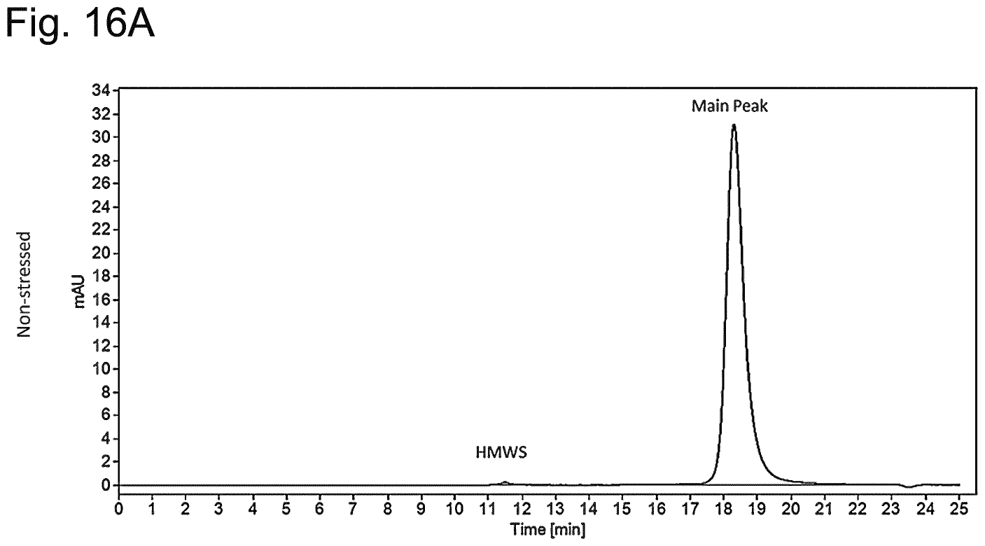

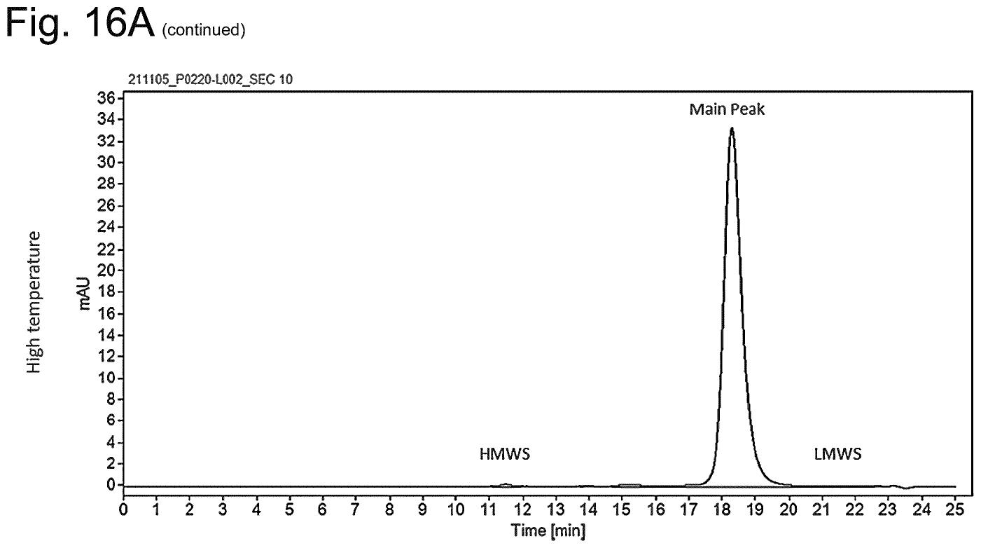

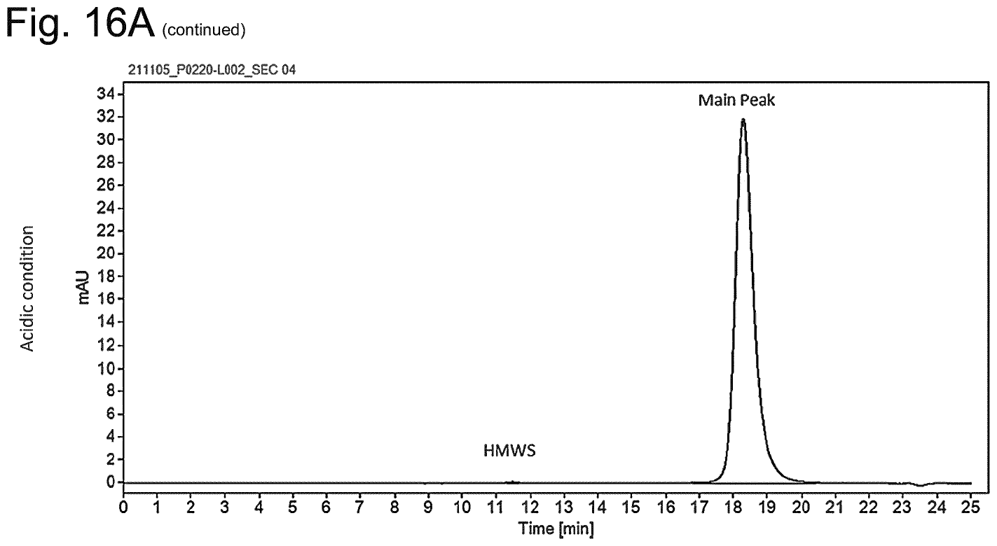

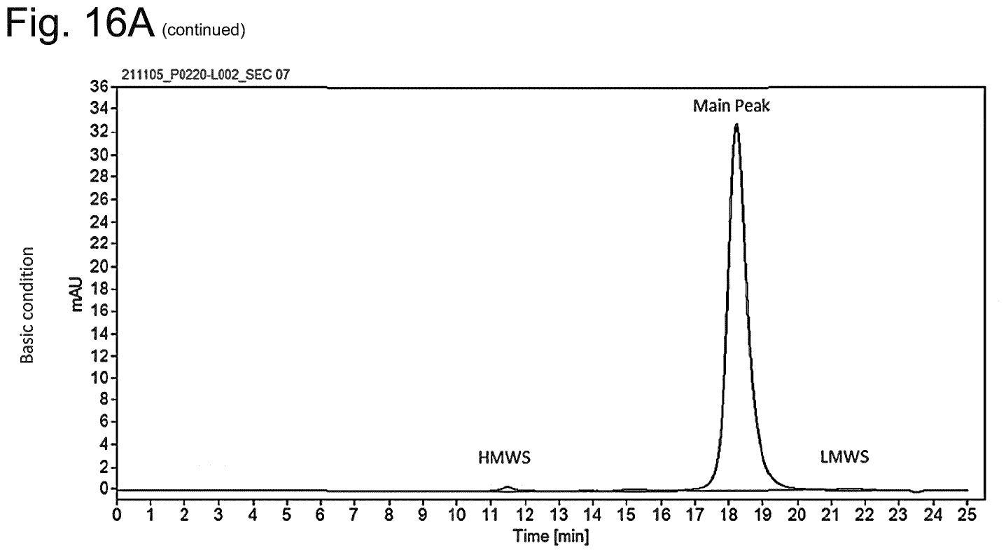

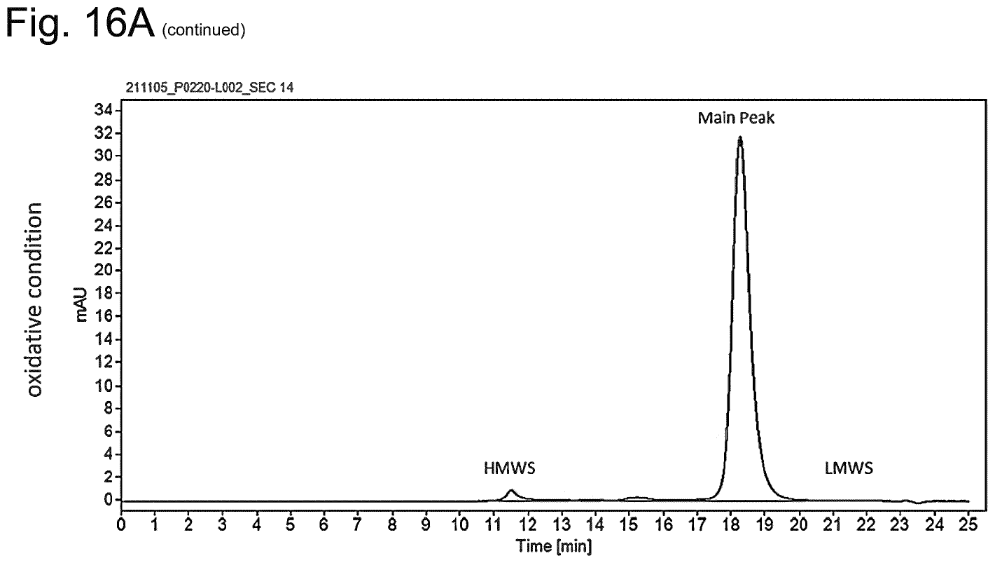

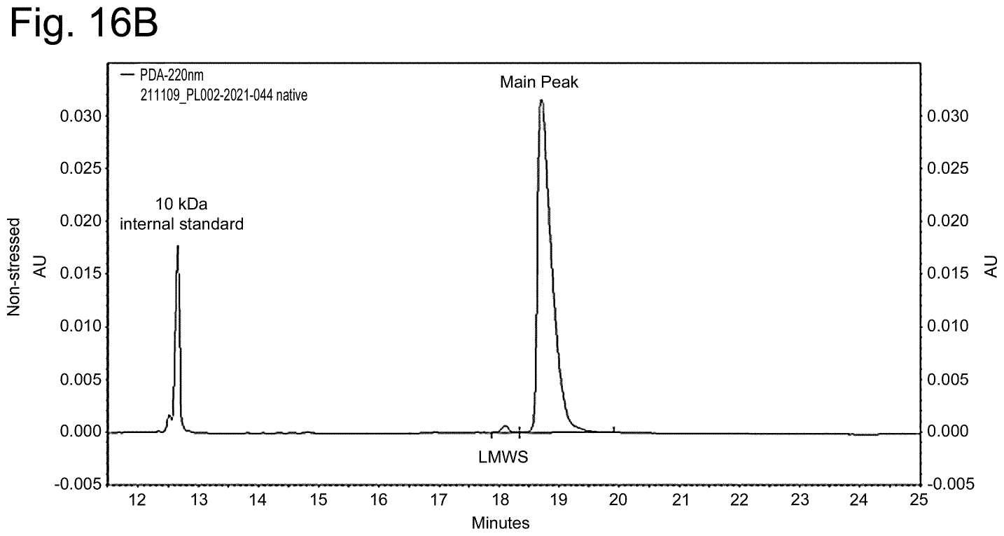

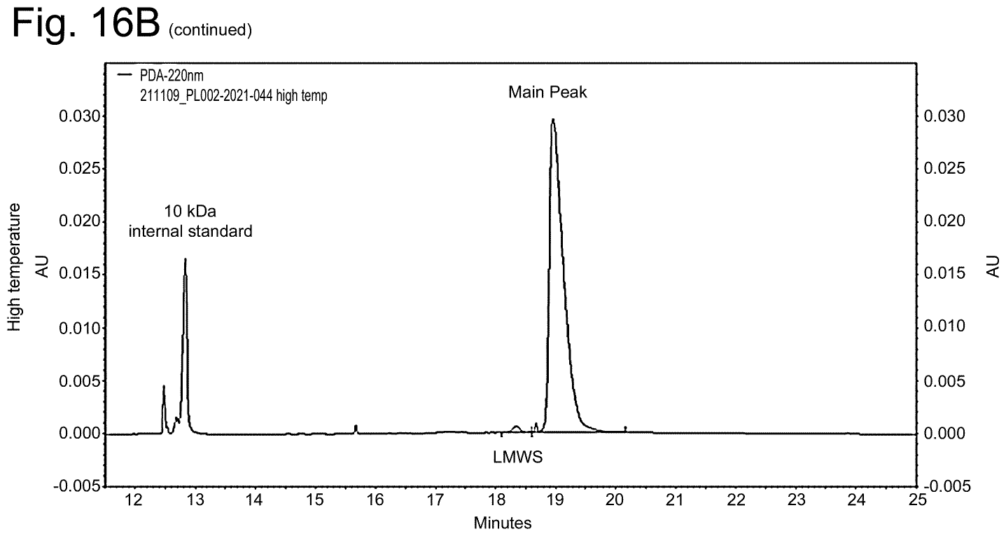

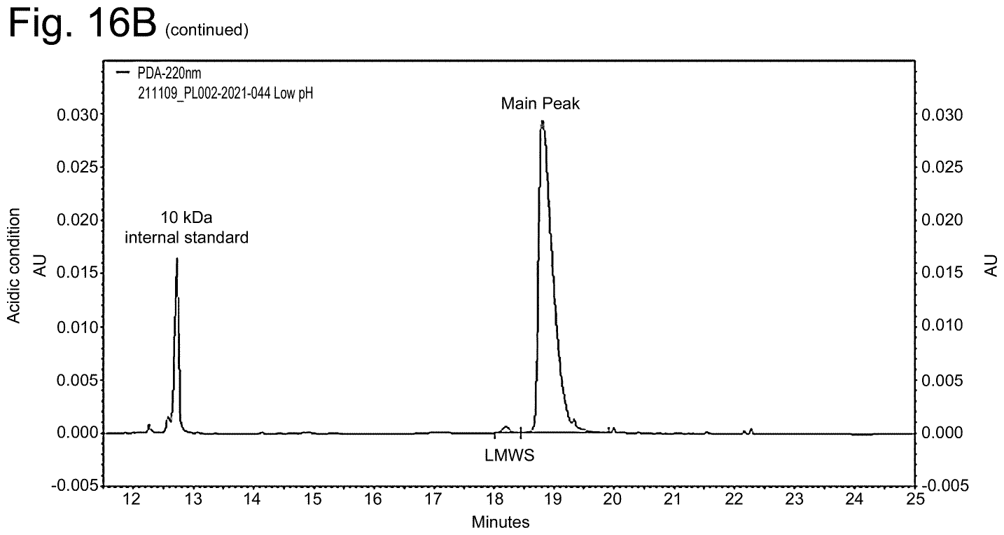

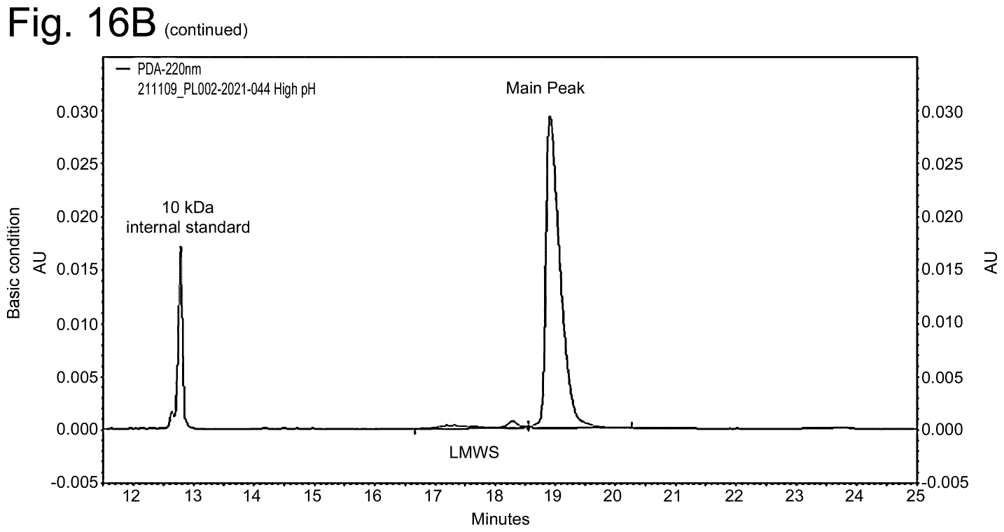

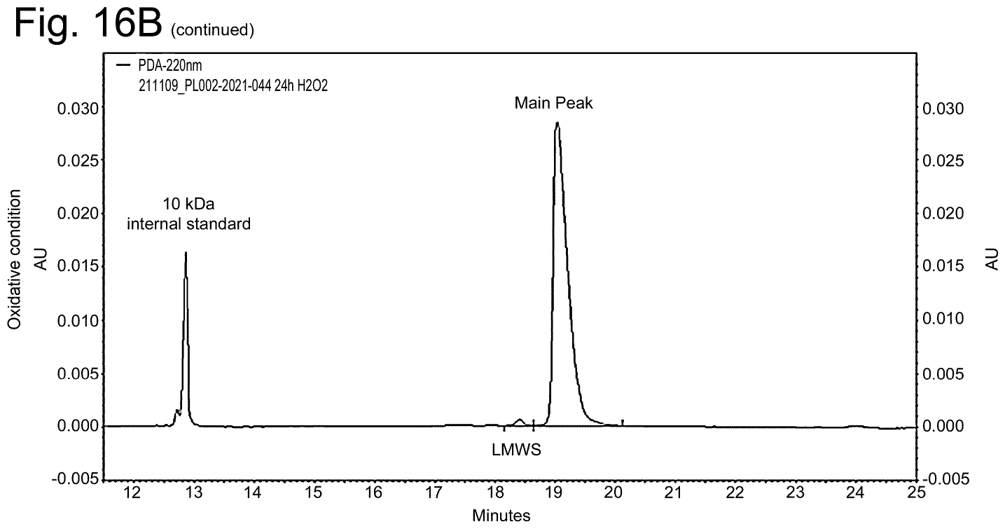

The thermal stability of 1D2x5C8var1(Y105F)-Fc was analyzed by nano-differential scanning fluorimetry (nano-DSF). The protein showed a high thermal stability with unfolding temperatures >60° C. (Table 3). Additionally, 1D2x5C8var1(Y105F)-Fc was subjected to accelerated stress tests. The sample was incubated for 1 week at elevated temperature (40° C.) and under acidic (50 mM acetate buffer, pH 5.0) and basic (100 mM phosphate buffer, pH 8.5) conditions, as well as 6 and 24 hours under oxidative conditions (phosphate buffer, pH 7.4 and 0.05% H₂O₂). Any stress-induced changes were analyzed by measuring aggregates and fragments by (A) size exclusion chromatography detected by ultraviolet absorption (SEC-UV) and (B) capillary gel electrophoresis under denaturing (SDS) conditions (CE-SDS) and after reduction (FIG. 16). No detectable degradation of the stressed protein sample in comparison to the non-stressed reference samples could be observed; the protein was found to be very stable.

TABLE 3

| | | | 1D2 × |
|---|---|---|---|
| Results nanoDSF | | | 5C8var1(Y105F)-Fc |
| Unfolding | ON [° C.] | Average | 60.45 |
| transition | | Standard deviation | 1.71 |
| observed at 350 | IP #1 [° C.] | Average | 65.65 |
| nm/Tm | | Standard deviation | 0.01 |
| | IP #2 [° C.] | Average | 71.61 |
| | | Standard deviation | 0.09 |

| Turbidity onset Tₒₙ | Average | 67.75 |
|---|---|---|
| | Standard deviation | 2.13 |

### Example 15: Vδ2-CD123 Bispecific Antibody 1D2x5C8var1(Y105F) is More Potent than a 7A5 Based Vγ9-CD123 Bispecific Antibody in Inducing T Cell Activation and T-Cell Mediated Tumor Cell Cytotoxicity

The potency of VO2-CD123 bispecific antibody 1D2x5C8var1(Y105F)-Fc was compared to a Vγ9-CD123-Fc bispecific antibody based on Vγ9-binding Fab antibody 7A5. The sequence of 7A5 was kindly provided by Prof. Kabelitz. The antibody has been characterized in Oberg et al. (2014) Cancer Res 74(5):1349.

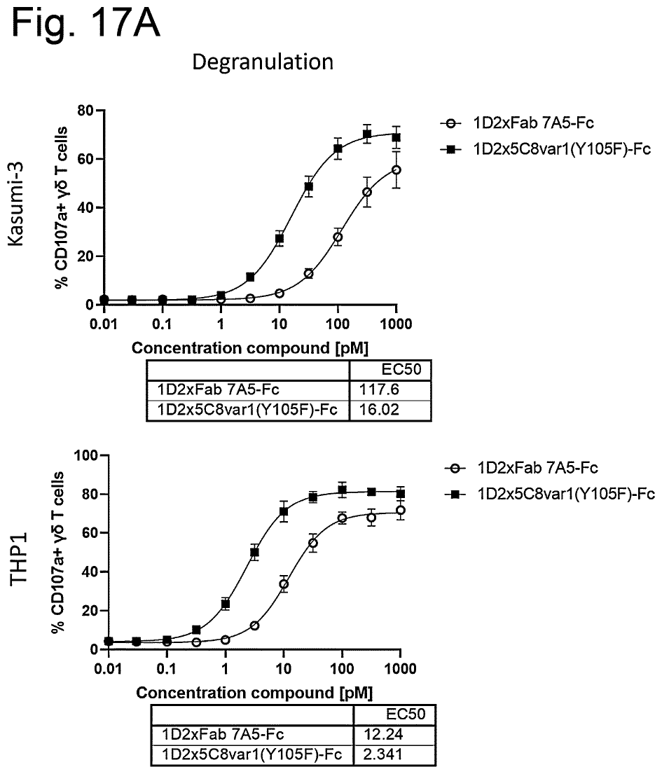

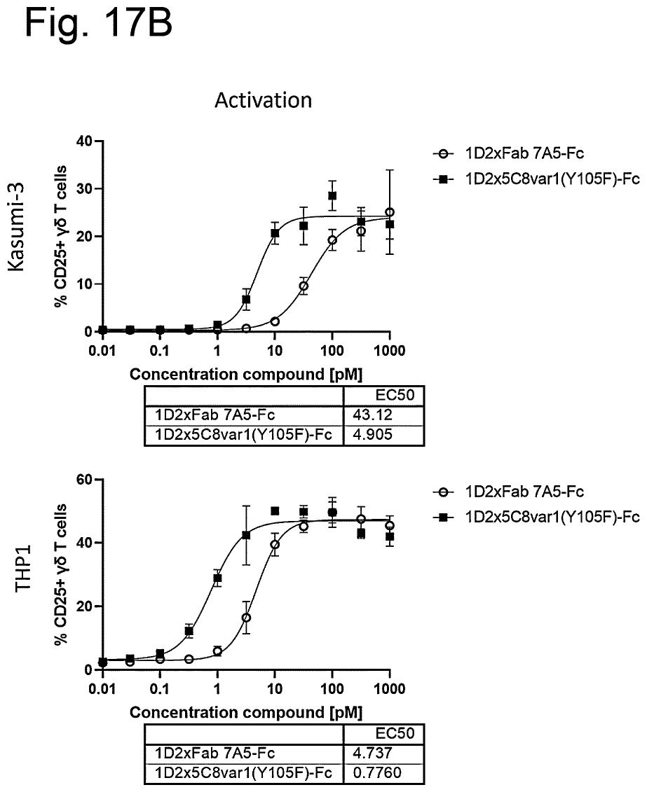

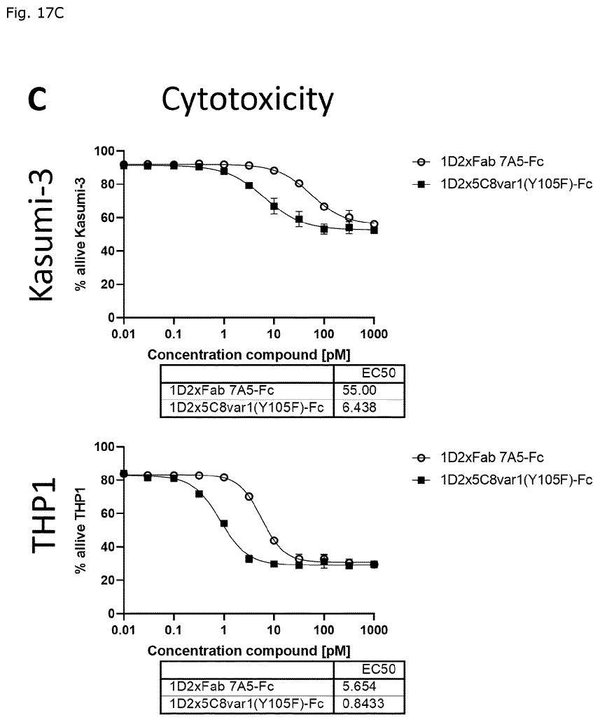

Variants of antibody 7A5 have been described in Ganesan et al. (2021) Leukemia 35(8):2274-2284 and WO2020/227457. The sequence of the Fab 7A5 was fused to a human CH2 and CH3 sequence including knob-into-hole mutations in CH3 and the Fc silencing mutations L234F and L235E. This Vγ9 binding ‘half-IgG’ (SEQ ID NO:51 and 52) was then co-expressed with the CD123 specific VHH-Fc fusion in HEK293E cells to form a bispecific Vγ9xCD123 bispecific antibody. The protein was purified over the C-terminal tag present at the C-terminus of the CH3 in the Vγ9 binding arm and then further purified via preparative size exclusion. This yielded pure and essentially endotoxin-free protein. 1D2xFab 7A5-Fc has the same CD123 binding VHH arm as 1D2x5C8var1(Y105F)-Fc. For the assay, 50,000 expanded Vγ9Vδ2 T cells were cocultured with 50,000 Kasumi-3 or THP1 target cells and a dilution series of the two compounds. Results are shown in FIG. 17. (A) Degranulation was analyzed after 4 hours by measuring the percentage CD107a (lysosomal-associated protein-1, or LAMP-1) positive cells via flow cytometry. (B) Vγ9Vδ2 T cell activation was analyzed by measuring the percentage CD25 positive cells and (C) cytotoxicity by analyzing the percentage live target cells after 24 hours via flow cytometry. 1D2x5C8var1(Y105F)-Fc induced a more potent activation and degranulation of the Vγ9Vδ2 T cells as witnessed by the higher percentage of CD107a positive cells as well as CD25 positive cells and lower EC50 value than those observed when using the Vγ9 targeting compound. A higher potency in cytotoxicity was observed using 1D2x5C8var1(Y105F)-Fc compared to 1D2xFab 7A5-Fc as proven by the significantly lower EC50 value.

1. A multispecific antibody comprising a first antigen-binding region capable of binding human CD123 and a second antigen-binding region capable of binding the Vδ2 chain of a human Vγ9Vδ2 T cell receptor.

2. The multispecific antibody according to claim 1, wherein the multispecific antibody is a bispecific antibody.

3. The multispecific antibody according to any one of the preceding claims, wherein the first antigen-binding region is a single-domain antibody and/or the second antigen-binding region is a single-domain antibody.

4. The multispecific antibody according to any one of the preceding claims, wherein the multispecific antibody competes for binding to human CD123 with an antibody having the sequence set forth in SEQ ID NO:1, preferably wherein the multispecific antibody binds the same epitope on human CD123 as an antibody having the sequence set forth in SEQ ID NO:1.

5. The multispecific antibody according to any one of the preceding claims, wherein the first antigen-binding region comprises the VH CDR1 sequence set forth in SEQ ID NO:2, the VH CDR2 sequence set forth in SEQ ID NO:3 and the VH CDR3 sequence set forth in SEQ ID NO:4, wherein preferably the first antigen-binding region comprises or consists of: a sequence selected from the group of sequences set forth in SEQ ID NO:1, 25 to 34, or a sequence having at least 90%, such as least 92%, e.g. at least 94%, such as at least 96%, e.g. at least 98% sequence identity to a sequence selected from the group of sequences set forth in SEQ ID NO:1, 25 to 34.

6. The multispecific antibody according to any one of claims 1 to 3, wherein the multispecific antibody competes for binding to human CD123 with an antibody having the sequence set forth in SEQ ID NO:9, preferably wherein the multispecific antibody binds the same epitope on human CD123 as an antibody having the sequence set forth in SEQ ID NO:9.

7. The multispecific antibody according to claim 6, wherein the first antigen-binding region comprises the VH CDR1 sequence set forth in SEQ ID NO:10, the VH CDR2 sequence set forth in SEQ ID NO:11 and the VH CDR3 sequence set forth in SEQ ID NO:12, wherein preferably the first antigen-binding region comprises or consists of: the sequence set forth in SEQ ID NO:9, or a sequence having at least 90%, such as least 92%, e.g. at least 94%, such as at least 96%, e.g. at least 98% sequence identity to the sequence set forth in SEQ ID NO:9.

8. The multispecific antibody according to any one of the preceding claims, wherein the multispecific antibody is able to activate human Vγ9Vδ2 T cells.

9. The multispecific antibody according to any one of the preceding claims, wherein the multispecific antibody competes for binding to human Vδ2 with an antibody having the sequence set forth in SEQ ID NO:17 wherein X₄ is Y, preferably wherein the multispecific antibody binds the same epitope on human Vδ2 as an antibody having the sequence set forth in SEQ ID NO:17 wherein X₄ is Y,
or
wherein the multispecific antibody competes for binding to human Vδ2 with an antibody having the sequence set forth in SEQ ID NO:36, preferably wherein the multispecific antibody binds the same epitope on human Vδ2 as an antibody having the sequence set forth in SEQ ID NO:36,
or
wherein the multispecific antibody competes for binding to human Vδ2 with an antibody having the sequence set forth in SEQ ID NO:37, preferably wherein the multispecific antibody binds the same epitope on human Vδ2 as an antibody having the sequence set forth in SEQ ID NO:37,
or
wherein the multispecific antibody competes for binding to human Vδ2 with an antibody having the sequence set forth in SEQ ID NO:38, preferably wherein the multispecific antibody binds the same epitope on human Vδ2 as an antibody having the sequence set forth in SEQ ID NO:38.

10. The multispecific antibody according to any one of the preceding claims, wherein the second antigen-binding region comprises the VH CDR1 sequence set forth in SEQ ID NO:18, the VH CDR2 sequence set forth in SEQ ID NO:19 and the VH CDR3 sequence set forth in SEQ ID NO:20, wherein preferably the second antigen-binding region comprises or consists of the sequence set forth in SEQ ID NO:17, or a sequence having at least 90%, such as least 92%, e.g. at least 94%, such as at least 96%, e.g. at least 98% sequence identity to the sequence set forth in SEQ ID NO:17,
or
wherein the second antigen-binding region comprises the VH CDR1 sequence set forth in SEQ ID NO:39, the VH CDR2 sequence set forth in SEQ ID NO:40 and the VH CDR3 sequence set forth in SEQ ID NO:41, wherein preferably the second antigen-binding region comprises or consists of the sequence set forth in SEQ ID NO:36, or a sequence having at least 90%, such as least 92%, e.g. at least 94%, such as at least 96%, e.g. at least 98% sequence identity to the sequence set forth in SEQ ID NO:36,
or
wherein the second antigen-binding region comprises the VH CDR1 sequence set forth in SEQ ID NO:42, the VH CDR2 sequence set forth in SEQ ID NO:43 and the VH CDR3 sequence set forth in SEQ ID NO:44, wherein preferably the second antigen-binding region comprises or consists of the sequence set forth in SEQ ID NO:37, or a sequence having at least 90%, such as least 92%, e.g. at least 94%, such as at least 96%, e.g. at least 98% sequence identity to the sequence set forth in SEQ ID NO:37,
or
wherein the second antigen-binding region comprises the VH CDR1 sequence set forth in SEQ ID NO:45, the VH CDR2 sequence set forth in SEQ ID NO:46 and the VH CDR3 sequence set forth in SEQ ID NO:47, wherein preferably the second antigen-binding region comprises or consists of the sequence set forth in SEQ ID NO:38, or a sequence having at least 90%, such as least 92%, e.g. at least 94%, such as at least 96%, e.g. at least 98% sequence identity to the sequence set forth in SEQ ID NO:38.

11. The multispecific antibody according to any one of the preceding claims, wherein
(i) the first antigen-binding region comprises the VH CDR1 sequence set forth in SEQ ID NO:2, the VH CDR2 sequence set forth in SEQ ID NO:3 and the VH CDR3 sequence set forth in SEQ ID NO:4 and the second antigen-binding region comprises the VH CDR1 sequence set forth in SEQ ID NO:18, the VH CDR2 sequence set forth in SEQ ID NO:19 and the VH CDR3 sequence set forth in SEQ ID NO:20, or
(ii) the first antigen-binding region comprises the VH CDR1 sequence set forth in SEQ ID NO:10, the VH CDR2 sequence set forth in SEQ ID NO:11 and the VH CDR3 sequence set forth in SEQ ID NO:12 and the second antigen-binding region comprises the VH CDR1 sequence set forth in SEQ ID NO:18, the VH CDR2 sequence set forth in SEQ ID NO:19 and the VH CDR3 sequence set forth in SEQ ID NO:20.

12. The multispecific antibody according to any one of the preceding claims, wherein the first antigen-binding region capable of binding human CD123 is located N-terminally of the second antigen-binding region capable of binding the human Vδ2 chain.

13. The multispecific antibody according to any one of the preceding claims, wherein the multispecific antibody further comprises a half-life extension domain, such as an Fc region, preferably a human Fc region.

14. The multispecific antibody according claim 13, wherein the Fc region is a heterodimer comprising two Fc polypeptides, wherein the first antigen-binding region is fused to the first Fc polypeptide and the second antigen-binding region is fused to the second Fc polypeptide and wherein the first and second Fc polypeptides comprise asymmetric amino acid mutations that favor the formation of heterodimers over the formation of homodimers, wherein preferably the first Fc polypeptide comprises a T366W substitution and the second Fc polypeptide comprises T366S, L368A and Y407V substitutions, or vice versa, wherein the amino acid positions correspond to human IgG1 according to the EU numbering system.

15. The multispecific antibody according any one of claim 13 or 14, wherein the cysteine residues at position 220 in the first and second Fc polypeptides have been deleted or substituted, wherein the amino acid position corresponds to human IgG1 according to the EU numbering system.

16. The multispecific antibody according any one of claims 13 to 15, wherein the first and second Fc polypeptides further comprise a mutation at position 234 and/or 235, preferably wherein the first and second Fc polypeptide comprise an L234F and an L235E substitution, wherein the amino acid positions correspond to human IgG1 according to the EU numbering system.

17. The multispecific antibody according to any one of the claims 13 to 16, wherein the first Fc polypeptide comprises the sequence set forth in SEQ ID NO:21 and the second Fc polypeptide comprises the sequence set forth in SEQ ID NO:22, or vice versa.

18. The multispecific antibody according to any one of the preceding claims, wherein the multispecific antibody is capable of mediating killing of CD123-expressing cells, such as C1r-neo cells or THP-1 cells, by Vγ9Vδ2 T cells.

19. An antibody comprising a first antigen-binding region capable of binding human CD123, wherein the first antigen-binding region is a single-domain antibody comprising:
(i) VH CDR1 sequence set forth in SEQ ID NO:2, the VH CDR2 sequence set forth in SEQ ID NO:3 and the VH CDR3 sequence set forth in SEQ ID NO:4, wherein preferably the first antigen-binding region comprises or consists of: a sequence selected from the group of sequences set forth in SEQ ID NO:1, 25 to 34, or a sequence having at least 90%, such as least 92%, e.g. at least 94%, such as at least 96%, e.g. at least 98% sequence identity to a sequence selected from the group of sequences set forth in SEQ ID NO:1, 25 to 34, or
(ii) the VH CDR1 sequence set forth in SEQ ID NO:10, the VH CDR2 sequence set forth in SEQ ID NO:11 and the VH CDR3 sequence set forth in SEQ ID NO:12, wherein preferably the first antigen-binding region comprises or consists of: the sequence set forth in SEQ ID NO:9, or a sequence having at least 90%, such as least 92%, e.g. at least 94%, such as at least 96%, e.g. at least 98% sequence identity to the sequence set forth in SEQ ID NO:9.

20. A pharmaceutical composition comprising a multispecific antibody according to any one of the preceding claims or the antibody according to claim 19 and a pharmaceutically-acceptable excipient.

21. The multispecific antibody according to any one of claims 1 to 18 or the antibody according to claim 19 for use as a medicament, preferably for use in the treatment of cancer, more preferably for use in the treatment of acute myeloid leukemia, B-cell acute lymphoblastic leukemia, hairy cell leukemia, Hodgkin lymphoma, blastic plasmacytoid dendritic neoplasm, chronic myeloid leukemia, chronic lymphocytic leukemia, B-cell chronic lymphoproliferative disorders or myelodysplastic syndrome.

22. A nucleic acid construct comprising a nucleotide sequence encoding an antibody of according to any one of claims 1 to 19 or a host cell comprising one or more nucleic acid constructs encoding an antibody according to any one of claims 1 to 19.


SEQUENCE LISTING (53 sequences)

SEQ ID NO: 1 - protein, 131 aa, Artificial Sequence
  antibody sequence
EVQLVESGGGLVQAGGSLRLSCAASGRTASSYVMGWFRQAPGKEREFVAVINWNGDSTYYTDSVKGRFAISRDNAKNTVYLQMNSLKPEDTAVYYCAADTRREWYRDGYWGPPARYEYDYRGQGTQVTVSS

SEQ ID NO: 2 - protein, 10 aa, Artificial Sequence
  antibody sequence
  VARIANT (10)..(10): Xaa is Gly or Ser
GRTASSYVMX

SEQ ID NO: 3 - protein, 17 aa, Artificial Sequence
  antibody sequence
  VARIANT (12)..(12): Xaa is Ala or Thr
VINWNGDSTYYXDSVKG

SEQ ID NO: 4 - protein, 22 aa, Artificial Sequence
  antibody sequence
  VARIANT (11)..(11): Xaa is Tyr or Phe
DTRREWYRDGXWGPPARYEYDY

SEQ ID NO: 5 - protein, 25 aa, Artificial Sequence
  antibody sequence
EVQLVESGGGLVQAGGSLRLSCAAS

SEQ ID NO: 6 - protein, 14 aa, Artificial Sequence
  antibody sequence
WFRQAPGKEREFVA

SEQ ID NO: 7 - protein, 32 aa, Artificial Sequence
  antibody sequence
RFAISRDNAKNTVYLQMNSLKPEDTAVYYCAA

SEQ ID NO: 8 - protein, 11 aa, Artificial Sequence
  antibody sequence
RGQGTQVTVSS

SEQ ID NO: 9 - protein, 122 aa, Artificial Sequence
  antibody sequence
EVQLVESGGGLVQAGGSLRLSCAASGRAINTYAMAWFRQAPGKERDFVATISYSGGTTDYAGSVKGRFTISRDNAENTVYLQMNSLKPEDTAVYYCAARDRYNPLARNYNYWGQGTQVTVSS

SEQ ID NO: 10 - protein, 10 aa, Artificial Sequence
  antibody sequence
GRAINTYAMA

SEQ ID NO: 11 - protein, 17 aa, Artificial Sequence
  antibody sequence
TISYSGGTTDYAGSVKG

SEQ ID NO: 12 - protein, 13 aa, Artificial Sequence
  antibody sequence
RDRYNPLARNYNY

SEQ ID NO: 13 - protein, 25 aa, Artificial Sequence
  antibody sequence
EVQLVESGGGLVQAGGSLRLSCAAS

SEQ ID NO: 14 - protein, 14 aa, Artificial Sequence
  antibody sequence
WFRQAPGKERDFVA

SEQ ID NO: 15 - protein, 32 aa, Artificial Sequence
  antibody sequence
RFTISRDNAENTVYLQMNSLKPEDTAVYYCAA

SEQ ID NO: 16 - protein, 11 aa, Artificial Sequence
  antibody sequence
WGQGTQVTVSS

SEQ ID NO: 17 - protein, 130 aa, Artificial Sequence
  antibody sequence
  VARIANT (105)..(105): Xaa is Tyr, Phe or Ser
EVQLLESGGGSVQPGGSLRLSCAASGRPFSNYAMSWFRQAPGKEREFVSAISWSGGSTSYADSVKGRFTISRDNSKNTLYLQMNSLRAEDTAVYYCAAQFSGADXGFGRLGIRGYEYDYWGQGTQVTVSS

SEQ ID NO: 18 - protein, 5 aa, Artificial Sequence
  antibody sequence
NYAMS

SEQ ID NO: 19 - protein, 17 aa, Artificial Sequence
  antibody sequence
AISWSGGSTSYADSVKG

SEQ ID NO: 20 - protein, 21 aa, Artificial Sequence
  antibody sequence
  VARIANT (7)..(7): Xaa is Tyr, Phe or Ser
QFSGADXGFGRLGIRGYEYDY

SEQ ID NO: 21 - protein, 217 aa, Artificial Sequence
  antibody sequence
APEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 22 - protein, 217 aa, Artificial Sequence
  antibody sequence
APEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 378 aa, Homo sapiens
MVLLWLTLLLIALPCLLQTKEDPNPPITNLRMKAKAQQLTWDLNRNVTDIECVKDADYSMPAVNNSYCQFGAISLCEVTNYTVRVANPPFSTWILFPENSGKPWAGAENLTCWIHDVDFLSCSWAVGPGAPADVQYDLYLNVANRRQQYECLHYKTDAQGTRIGCRFDDISRLSSGSQSSHILVRGRSAAFGIPCTDKFVVFSQIEILTPPNMTAKCNKTHSFMHWKMRSHFNRKFRYELQIQKRMQPVITEQVRDRTSFQLLNPGTYTVQIRARERVYEFLSAWSTPQRFECDQEEGANTRAWRTSLLIALGTLLALVCVFVICRRYLVMQRLFPRIPHMKDPIGDSFQNDKLVVWEAGKAGLEECLVTEVQVVQKT

SEQ ID NO: 24 - protein, 897 aa, Homo sapiens
MVLAQGLLSMALLALCWERSLAGAEETIPLQTLRCYNDYTSHITCRWADTQDAQRLVNVTLIRRVNEDLLEPVSCDLSDDMPWSACPHPRCVPRRCVIPCQSFVVTDVDYFSFQPDRPLGTRLTVTLTQHVQPPEPRDLQISTDQDHFLLTWSVALGSPQSHWLSPGDLEFEVVYKRLQDSWEDAAILLSNTSQATLGPEHLMPSSTYVARVRTRLAPGSRLSGRPSKWSPEVCWDSQPGDEAQPQNLECFFDGAAVLSCSWEVRKEVASSVSFGLFYKPSPDAGEEECSPVLREGLGSLHTRHHCQIPVPDPATHGQYIVSVQPRRAEKHIKSSVNIQMAPPSLNVTKDGDSYSLRWETMKMRYEHIDHTFEIQYRKDTATWKDSKTETLQNAHSMALPALEPSTRYWARVRVRTSRTGYNGIWSEWSEARSWDTESVLPMWVLALIVIFLTIAVLLALRFCGIYGYRLRRKWEEKIPNPSKSHLFQNGSAELWPPGSMSAFTSGSPPHQGPWGSRFPELEGVFPVGFGDSEVSPLTIEDPKHVCDPPSGPDTTPAASDLPTEQPPSPQPGPPAASHTPEKQASSFDFNGPYLGPPHSRSLPDILGQPEPPQEGGSQKSPPPGSLEYLCLPAGGQVQLVPLAQAMGPGQAVEVERRPSQGAAGSPSLESGGGPAPPALGPRVGGQDQKDSPVAIPMSSGDTEDPGVASGYVSSADLVFTPNSGASSVSLVPSLGLPSDQTPSLCPGLASGPPGAPGPVKSGFEGYVELPPIEGRSPRSPRNNPVPPEAKSPVLNPGERPADVSPTSPQPEGLLVLQQVGDYCFLPGLGPGPLSLRSKPSSPGPGPEIKNLDQAFQVKKPPGQAVPQVPVIQLFKALKQQDYLSLPPWEVNKPGEVC

SEQ ID NO: 25 - protein, 131 aa, Artificial Sequence
  antibody sequence
EVQLVESGGGLVQPGGSLRLSCAASGRTASSYVMGWFRQAPGKEREFVSVINWNGDSTYYADSVKGRFTISRDNSKNTLYLQMNSLRAEDTAVYYCAADTRREWYRDGYWGPPARYEYDYRGQGTQVTVSS

SEQ ID NO: 26 - protein, 131 aa, Artificial Sequence
  antibody sequence
EVQLVESGGGLVQPGGSLRLSCAASGRTASSYVMGWVRQAPGKEREWVSVINWNGDSTYYADSVKGRFTISRDNSKNTLYLQMNSLRAEDTAVYYCAADTRREWYRDGYWGPPARYEYDYRGQGTLVTVSS

SEQ ID NO: 27 - protein, 131 aa, Artificial Sequence
  antibody sequence
EVQLVESGGGLVQPGGSLRLSCAASGRTASSYVMSWFRQAPGKEREWVAVINWNGDSTYYADSVKGRFTISRDNSKNTVYLQMNSLRAEDTAVYYCAADTRREWYRDGYWGPPARYEYDYRGQGTLVTVSS

SEQ ID NO: 28 - protein, 131 aa, Artificial Sequence
  antibody sequence
EVQLVESGGGVVQPGGSLRLSCAASGRTASSYVMSWFRQAPGKEREWVAVINWNGDSTYYADSVKGRFTISRDNSKNTLYLQMNSLRAEDTAVYYCAADTRREWYRDGYWGPPARYEYDYRGQGTTVTVSS

SEQ ID NO: 29 - protein, 131 aa, Artificial Sequence
  antibody sequence
EVQLVESGGGLVQPGGSLRLSCAASGRTASSYVMSWFRQAPGKEREFVAVINWNGDSTYYADSVKGRFTISRDNSKNTVYLQMNSLRAEDTAVYYCAADTRREWYRDGYWGPPARYEYDYRGQGTLVTVSS

SEQ ID NO: 30 - protein, 131 aa, Artificial Sequence
  antibody sequence
EVQLVESGGGLVQPGGSLRLSCAASGRTASSYVMGWVRQAPGKGLEWVSVINWNGDSTYYADSVKGRFTISRDNSKNTLYLQMNSLRAEDTAVYYCAADTRREWYRDGYWGPPARYEYDYRGQGTLVTVSS

SEQ ID NO: 31 - protein, 131 aa, Artificial Sequence
  antibody sequence
EVQLVESGGGLVQPGGSLRLSCAASGRTASSYVMSWVRQAPGKGLEWVSVINWNGDSTYYADSVKGRFTISRDNSKNTLYLQMNSLRAEDTAVYYCAADTRREWYRDGYWGPPARYEYDYRGQGTLVTVSS

SEQ ID NO: 32 - protein, 131 aa, Artificial Sequence
  antibody sequence
EVQLVESGGGLVQPGGSLRLSCAASGRTASSYVMSWFRQAPGKGLEWVAVINWNGDSTYYADSVKGRFTISRDNSKNTVYLQMNSLRAEDTAVYYCAADTRREWYRDGYWGPPARYEYDYRGQGTLVTVSS

SEQ ID NO: 33 - protein, 131 aa, Artificial Sequence
  antibody sequence
EVQLVESGGGLVQAGGSLRLSCAASGRTASSYVMGWFRQAPGKEREWVAVINWNGDSTYYTDSVKGRFAISRDNAKNTVYLQMNSLRAEDTAVYYCAADTRREWYRDGYWGPPARYEYDYRGQGTQVTVSS

SEQ ID NO: 34 - protein, 131 aa, Artificial Sequence
  antibody sequence
EVQLVESGGGLVQPGGSLRLSCAASGRTASSYVMGWFRQAPGKEREFVSVINWNGDSTYYADSVKGRFTISRDNSKNTLYLQMNSLRAEDTAVYYCAADTRREWYRDGFWGPPARYEYDYRGQGTQVTVSS

SEQ ID NO: 35 - protein, 14 aa, Artificial Sequence
  antibody sequence
AAASDKTHTCPPCP

SEQ ID NO: 36 - protein, 126 aa, Artificial Sequence
  antibody sequence
EVQLVESGGGLVQAGGSLRLSCAASGRPFSNYGMGWFRQAPGKKREFVAGISWSGGSTDYADSVKGRFTISRDNAKNTVYLQMNSLKPEDTAVYYCAAVFSGAETAYYPSDDYDYWGQGTQVTVSS

SEQ ID NO: 37 - protein, 126 aa, Artificial Sequence
  antibody sequence
EVQLVESGGGLVQAGGSLRLSCAASGRPFSNYGMGWFRQAPGKKRESVAGISWSGGSTDYADSVKGRFTISRDNAKNTVYLQMNSLKPEDTAVYYCAAVFSGAETAYYPSDDYDYWGQGTQVTVSS

SEQ ID NO: 38 - protein, 127 aa, Artificial Sequence
  antibody sequence
EVQLVESGGGLVQAGGSLRLSCAASGRPFSNYAMGWFRQAPGKEREFVTVISWSGGSTYYADSVKGRFTISRDNAKNTVYLQMNSLKPEDTAVYYCAAQFSGASTVVAGTALDYDYWGQGTRVTVSS

SEQ ID NO: 39 - protein, 10 aa, Artificial Sequence
  antibody sequence
GRPFSNYGMG

SEQ ID NO: 40 - protein, 17 aa, Artificial Sequence
  antibody sequence
GISWSGGSTDYADSVKG

SEQ ID NO: 41 - protein, 17 aa, Artificial Sequence
  antibody sequence
VFSGAETAYYPSDDYDY

SEQ ID NO: 42 - protein, 10 aa, Artificial Sequence
  antibody sequence
GRPFSNYGMG

SEQ ID NO: 43 - protein, 17 aa, Artificial Sequence
  antibody sequence
GISWSGGSTDYADSVKG

SEQ ID NO: 44 - protein, 17 aa, Artificial Sequence
  antibody sequence
VFSGAETAYYPSDDYDY

SEQ ID NO: 45 - protein, 10 aa, Artificial Sequence
  antibody sequence
GRPFSNYAMG

SEQ ID NO: 46 - protein, 17 aa, Artificial Sequence
  antibody sequence
VISWSGGSTYYADSVKG

SEQ ID NO: 47 - protein, 18 aa, Artificial Sequence
  antibody sequence
QFSGASTVVAGTALDYDY

SEQ ID NO: 48 - protein, 267 aa, Homo sapiens
MQRISSLIHLSLFWAGVMSAIELVPEHQTVPVSIGVPATLRCSMKGEAIGNYYINWYRKTQGNTMTFIYREKDIYGPGFKDNFQGDIDIAKNLAVLKILAPSERDEGSYYCACDTLGMGGEYTDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSCHKPKAIVHTEKVNMMSLT

SEQ ID NO: 49 - protein, 361 aa, Artificial Sequence
  antibody sequence
EVQLLESGGGSVQPGGSLRLSCAASGRPFSNYAMSWFRQAPGKEREFVSAISWSGGSTSYADSVKGRFTISRDNSKNTLYLQMNSLRAEDTAVYYCAAQFSGADFGFGRLGIRGYEYDYWGQGTQVTVSSAAASDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - protein, 362 aa, Artificial Sequence
  antibody sequence
EVQLVESGGGLVQAGGSLRLSCAASGRTASSYVMGWFRQAPGKEREFVAVINWNGDSTYYTDSVKGRFAISRDNAKNTVYLQMNSLKPEDTAVYYCAADTRREWYRDGYWGPPARYEYDYRGQGTQVTVSSAAASDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 51 - protein, 455 aa, Artificial Sequence
  antibody sequence
QVQLQQSGAELARPGASVKLSCKASGFTFTDHYINWVKQRTGQGLEWIGQIYPGNGNTYYNEKFKGKATLTADKSSSTAYMQLSSLTSEDSAVYFCAPNYGDYTLDFWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKAAAEPEA

SEQ ID NO: 52 - protein, 219 aa, Artificial Sequence
  antibody sequence
DIVMSQSPSSLAVSVGEKVTMSCKSSQSLLYSSNQKNYLAWYQQKPGQSPKLLIYWASTRESGVPDRFTGSGSGTDFTLTISSVKAEDLAVYYCQQYYRYHTFGTGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 53 - protein, 7 aa, Artificial Sequence
  tag sequence
AAAEPEA